(19) United States
(12) Patent Application Publication
(10) Pub. No.: US 2011/0179841 A1
(43) Pub. Date: Jul. 28, 2011

(54) HIGH-RATE COMPOSTING PROCESS AND EQUIPMENT

(76) Inventor: James Cheng-Shyong Lu, Huntington Beach, CA (US)

(21) Appl. No.: 12/694,842

(22) Filed: Jan. 27, 2010

(51) Int. Cl. C05F 7/00; C12M 1/02

(52) U.S. Cl. 71/12; 435/290.2

(57) ABSTRACT

The present invention addresses methods and equipments for a high-rate production and improvement of the quality of composts from organic wastes, by processes including Screening, Mechanical Pretreatment, Physical-Chemical Pretreatment, High-Rate Stabilization Process, High-Rate Activation Process, and Product Refining Processes, and adding seven types of chemical agents including: Wetting Agents, Debonding Agents, Organic Stabilization Agents, Inorganic Stabilization Agents, Fluffing Agents, Activation Agents, and Nutrient Agents in order to enhance six major properties which activated characteristics of composts: (a) moisture absorption and holding capability, (b) nutrients adsorption and holding capability, (c) soil particles holding and conserving capability, (d) soil air ventilation capability, (e) soil water transmission capability, and (f) soil thermal insulation capability, as well as other minor quality improvements such as complete sterilization of pathogens and parasites, detoxification of many hazardous chemical species, and removal of excess heavy metal contents, if any, from the activated composting products.

### BACKGROUND OF THE INVENTION

1. Field of the Invention

This invention relates to a high-rate thermal-chemical-mechanical process and equipment for the production of high quality composts. This invention can convert organic wastes into high quality composts within about one hour. Types of organic wastes can be used for producing composts are municipal solid wastes (MSW), agricultural wastes, green wastes, wastewater treatment plant sludge, animal wastes, some types of organic wastes from manufacturing plants such as food processing, paper manufacturing, refinery, and medicine manufacturing plants, organic wastes from institutional facilities, as well as wastes from landfill mining (cleanup), etc. Comparing to the traditional bio-chemical composts produced, composts produced by the subject invention can achieve higher compost qualities such as with much higher water absorbing and holding capacities, higher nutrients (N, P, K) holding and long-term supply capabilities, higher adsorption capabilities for micro-nutrients, and achieving better air circulating, moisture transmission, and thermal insulation characteristics when mixed with soils. Composts produced by the subject invention also can achieve much better characteristics such as complete sterilization of pathogens and parasites, detoxification of many hazardous chemical species, and removal of excess heavy metal contents, if any, from the composting products.

2. Description of the Background Art

Benefits of applying organic composts to soils for growing agricultural crops or gardening plants have been widely reported and are known in the art. In general, major benefits can be summarized into four categories:
Nutrient Holding and Long-Term Supply Capabilities: Chelating effects of organic contents, especially cellulosic and humic substances, in composts can enhance the capabilities of soils for holding and long-term releasing of nutrients for the use by vegetation. This effect also can prevent migration of nutrients (mainly N, P, K compounds) from soils into the environment, therefore, reducing pollution problems. Organic acids generated from composts also could release micronutrients for the use by vegetation.
High Water Absorbing and Holding Capabilities: Again, properly treated cellulosic and humic substances in composts have very high water absorbing and holding capabilities, which can greatly improve the “Field Capacity” and “Available Water” in soils, therefore, improving crop growing rates and production amounts, and enhancing resistance capacity to drought.
Improving Soil Physical Structure: Organic contents, especially organocellulose derived fibers in composts, can effectively improve physical structure of soils such as increase air and water circulating and insulation characteristics, as a result, improving growing environments for roots, increasing gemmating rates of seeds. The organic fibers in composts also can reduce water and/or wind erosion loss of silts and clay particles, and avoid hardening of soils due to long-term use of synthetic fertilizers.
Stimulating Growth of Vegetation and Improving Crop Quality: Certain effects, although still in the research stage, such as stimulating effects of humic and fulvic acids, and biosurfactants generated in composts could improve the growth rate and quality of products produced.
   Due to the above listed advantages, production and application of composts in the agriculture field have been practiced way beyond our records by our ancestors. The relative primitive compost production methods recorded in history were over four thousand years ago (refer to U.S. Pat. No. 5,534,437, 1996). Since then, numerous improvements in compost production processes have been developed. These traditional composting methods are mainly based on biochemical reactions, using microorganisms to degrade organic wastes under certain favorable conditions of C/N ratio, temperature, moisture, and air circulating status causing “easily decomposable organics” to mostly decompose and release small amount of nutrients from organic wastes. These processes also may cause a small amount of certain “moderately biodegradable organics” to transform into various humic substances. One of the major drawbacks of traditional composting methods is the lengthy time needed for decomposition and curing of the organic wastes. In general, these processes need weeks to months to complete the compost production. If the compost production periods cannot be reduced to within one day or less, then this organic treatment method will not be able to become one of the “main stream” methods for treating daily large quantity of organic wastes generated, such as use for recycling current large quantity of municipal solid wastes and sludge generated. If a high rate composting process, such as only with hours of compost generating periods, can be developed, then replacement of the current two main stream solid wastes management methods—sanitary landfills and incineration by composting will become possible. In recent decades, improvement methods were invented with the objective to reduce the degradation and curing periods. These improvements were mainly based on (1) selection of equipment for dynamic or rigorously mixing to improve the contact opportunities between microorganisms and organics, (2) selection of the proper C/N ratio of the input wastes to closer to the C/N ratio of microorganisms, (3) control of moisture contents, (4) control of oxygen supply, and/or (5) selection of special types of microorganisms, enzymes, or chemicals to enhance the degradation of organics. Examples of the above improvement methods can be shown by the following patents: U.S. Pat. No. 4,062,770, 1977; U.S. Pat. No. 4,067,504, 1978; U.S. Pat. No. 4,483,704, 1984; U.S. Pat. No. 5,534,437, 1996; U.S. Pat. No. 6,648,940B2, 2003; Chinese patents CN1035654, 1989; CN2063940, 1990; CN2253347, 1997; CN2823257, 2006; CN101033154, 2007; CN201165498, 2008, etc. Even with the above improvements, none of the traditional composting processes can really generate composts within hours.

The above improvements were mainly based on degradation rate enhancements. Another type of improvement which is greatly lacking is the improvement of compost quality, such as enhancing the capacities of nutrients adsorption, moisture absorption and holding, impurity elimination, toxic compounds (such as heavy metals, toxic organics) removal, pathogens and parasites sterilization, and enhancement of the soil structure improvements (such as air circulation, moisture movement, heat insulation capacities) for growing vegetations, etc. In order to achieve the above mentioned compost production rates and higher qualities of products, traditional, or bio-chemical, production processes may not be able to accomplish the objectives. Therefore, development of new, non-traditional production processes becomes necessary.

The subject invention provides a new and non-traditional approach for compost production. The subject invention is not using the time-consuming microorganisms for compost generation, rather, it involves thermal-chemical-mechanical processes to “stabilize” the “easily decomposable organics” and to “activate” the remaining “moderately decomposable organics” to compounds with relative high capacities of nutrients adsorption, moisture absorption and holding, impurity elimination, toxic compounds (such as heavy metals, toxic organics) removal, pathogens and parasites sterilization, and enhancement of the soil structure improvements for growing vegetations, etc. The “easily decomposable organics” such as proteins, carbohydrates, sugars and fats are usually those organic compounds causing organic wastes becoming putrescible and causing nuisances, disease-carrying, parasites-carrying, and other pollutant-releasing sources. The subject invention creates relative high temperature, high pressure, aqueous phase, and partial oxidizing environments with the help of mechanical and oxidizing agents to “stabilize” those “easily decomposable organics” within tens of minutes. Certain toxic organic compounds can also be degraded under such environments within tens of minutes. Heavy metals, if present beyond limits in composts, can also be stabilized in the same reactor by using certain chemicals to be discussed later in this invention. The “moderately decomposable organics” such as lignocelluloses and their disassociation compounds (i.e., mainly celluloses, hemicelluloses, and lignin) in the MSW and other types of organic wastes, are further treated to release their cellulosic compounds and partially converting them to fibrous and humic substances by further steam and chemical treatment to “activate” those characteristics which causing the treated materials to become high quality composts. By doing so, the above stated high production rate and higher qualities of composts can be achieved.

There are thermal-chemical-mechanical processes which have been employed for organic wastes treatment, such as incineration, wet air oxidation, pyrolysis, hydrolysis, steam treatment, gasification, etc. Almost all of the above mentioned processes are focused on organic treatment/decomposition and/or energy production objectives, not on compost production purposes, especially when high quality of composts production are concerned. Among the above processes, only certain principles of wet air oxidation and steam explosion processes are close to part of the principles used by the subject invention.

The wet air oxidation process is a “complete” oxidation process by using relative high temperatures and pressures (usually in the range of 150 to 320° C., or about 300 to 610° F. for temperatures, and 150 to 3,200 psia for pressures) and enough dissolved oxygen with the objective to decompose organics to the maximum extents possible. Examples of patents related to wet air oxidation processes used for organic wastes treatment are U.S. Pat. No. 2,665,249, 1954; U.S. Pat. No. 3,060,118, 1962; U.S. Pat. No. 3,272,740, 1966; U.S. Pat. No. 3,870,631, 1975; U.S. Pat. No. 4,010,098, 1977; etc. However, the subject invention is using a “partial” oxidation process at relative lower temperatures and pressures and relative lower dissolved oxidant contents to decompose mainly the portion of the wastes in the category of “easily decomposable organics”. The “moderately decomposable organics” are mostly not reacted. Complete oxidation approach would reduce greatly the quantity of compost to be produced, which would reduce the economic value of the composting products and is not the objective of this method.

The steam explosion and the steam only (without explosion) processes have been used for MSW and other organic wastes treatment mainly for the purposes of separation of waste components for recycling or preparation of materials which would be more favorable or more amenable for further energy production or composting. Examples of related prior arts are shown in Table 1, as listed below. Brief comparisons and comments between these prior arts and the subject invention are also provided in Table 1 for references. As shown in the table, these steam treatment processes are seldom used directly to generate composts due to the fact that the team treatment processes are usually conducted in a reducing environment which “easily decomposable organics” cannot be totally oxidized and release nutrients. Some of the valuable nutrient components could be lost into the gas phase (such as converting nitrogen compounds to ammonia) due to reducing environment used in the steam treatment processes. However, steam explosion process is considered one of the most effective processes to disassociate lignocelluloses to celluloses, hemicelluloses, and lignin. The subject invention applying this process with the assistance of chemicals to further enhance the qualities of composts produced.

TABLE 1
Related Prior Arts employing Steam Treatment for Organic Wastes Recycling

| PRIOR ART | | METHODS/ | STEAM | |
|---|---|---|---|---|
| PATENT NO./ | INVENTER/ | PROCESS/ | EXPLOSION | COMPARISONS/ |
| (Title) | DATE | PURPOSES | USED | COMMENTS |
| U.S. Pat. No. 3,932,166 | Martin | Method: A method for | No | This prior art using |
| (Landfill and Soil | Vignovich, | converting organic | | acid addition and |
| Conditioner) | Russell Sperry | materials into inert | | heating to char the |
| | Jan. 13, 1976 | humus-like materials. | | organic wastes |
| | | Process: Heating, | | would need a |
| | | drying in the presence | | significant amount |
| | | of certain-soluble | | of external energy. |
| | | inorganic acids, then | | However, the |
| | | washing with water. | | subject invention |
| | | Purposes: Humus-like | | uses the energy |
| | | char reacted with an | | generated from the |
| | | alkali at elevated | | oxidation of |
| | | temperature, to be | | wastes. |
| | | used as soil | | This prior art could |
| | | conditioner. | | cause valuable |
| | | | | nutrient contents |
| | | | | and acid used |
| | | | | converting to gases |
| | | | | and cause air |
| | | | | pollution problems. |
| | | | | This prior art uses |
| | | | | strong acid and |
| | | | | base at high |
| | | | | temperatures |
| | | | | would need special |
| | | | | corrosion proof |
| | | | | materials for |
| | | | | equipments. |
| U.S. Pat. No. 4,056,380 | E. Brandt | Method/Process/ | No | 45 days of aerobic |
| (Method of Producing | Thiac | Purpose: Using | | decomposition are |
| An Organic Soil | Nov. 1, 1977 | aerobic (heap | | needed by this |
| Additive and the | | aeration) method to | | prior art to degrade |
| Product Thereof) | | degrade water | | organics. However, |
| | | hyacinths and sewage | | the subject |
| | | sludge mixtures into | | invention only need |
| | | compost, then using | | about one hour for |
| | | steam treatment to | | compost |
| | | produce a product | | production. |
| | | with high moisture | | |
| | | retention soil additive. | | |
| U.S. Pat. No. 4,342,830 | Clifford | Method/Process: | Yes | This prior art uses |
| (Process for | Holloway | Using steam | | steam explosion to |
| Separating and | Aug. 3, 1982 | explosion to sterilize | | recover organics |
| Recovering Organics | | and soften organics. | | from wastes, then |
| and Inorganics from | | Purposes: Using | | uses fermentation |
| Waste Material) | | steam explosion to | | to produce fuels, |
| | | separate organics | | and animal feed |
| | | from inorganics, then, | | supplements. |
| | | further processing of | | However, the |
| | | the materials to | | subject invention |
| | | recover fuels and | | uses steam |
| | | animal feed | | explosion to |
| | | supplements. | | generate more |
| | | | | cellulosic fibers |
| | | | | and humic |
| | | | | substances. |
| | | | | This prior art needs |
| | | | | external steam |
| | | | | generation. The |
| | | | | subject invention |
| | | | | uses steam |
| | | | | generated from the |
| | | | | wastes, so no |
| | | | | external energy is |
| | | | | needed for steam |
| | | | | supply. |
| U.S. Pat. No. 4,461,648 | Patrick Foody | Method/Process: | Yes | This prior art needs |
| (Method for | Jul. 24, 1984 | Lignocellulosic | | external steam |
| Increasing the | | materials are steam | | generation. The |
| Accessibility of | | cooked and then | | subject invention |
| Cellulose in | | rapidly | | uses steam |
| Lignocellulosic | | depressurized. | | generated from the |
| Materials, Particularly | | Venting volatiles are | | wastes, so no |
| Hardwoods | | used before steam | | external energy is |
| Agricultural Residues | | decompression. | | needed for steam |
| and the Like) | | Purpose: This | | supply. |
| | | method mainly uses | | This prior art uses |
| | | for increasing the | | steam explosion |
| | | accessibility of | | mainly for |
| | | cellulose to chemical | | increasing the |
| | | or biochemical | | accessibility of |
| | | reagents. | | cellulose to |
| | | | | chemical or |
| | | | | biochemical |
| | | | | reagents. However, |
| | | | | the subject |
| | | | | invention is mainly |
| | | | | using steam |
| | | | | explosion to |
| | | | | increase the quality |
| | | | | of composts. |
| U.S. Pat. No. 4,540,467 | Kenneth | Method/Process: | Yes | This prior art is |
| (Method for | Grube; | Using heating/ | | used to fragment |
| Fragmenting | Vincent | pressurization and | | and separate MSW |
| Municipal Solid | Harrington; | hydrating on municipal | | for further |
| Wastes) | James | solid wastes (MSW) to | | recycling and |
| | Harrington | soften the material. | | composting. |
| | Sep. 10, 1985 | Then liquid in the | | However, the |
| | | material is drained | | subject invention is |
| | | and pressurized | | used directly for |
| | | steam is added for | | compost |
| | | steam explosion | | production. |
| | | treatment to fragment | | This prior art needs |
| | | the material. | | external steam |
| | | Purposes: to fragment | | generation. The |
| | | MSW to replace the | | subject invention |
| | | current grinding | | uses steam |
| | | process used for size | | generated from the |
| | | reduction. | | wastes, so no |
| | | | | external energy is |
| | | | | needed for steam |
| | | | | supply. |
| U.S. Pat. No. 4,540,495 | Clifford | Method/Process: Heat | No | This prior art is for |
| (Process for Treating | Holloway | and pressure are used | | treating MSW in |
| Municipal Solid | Sep. 10, 1985 | to cook, sterilize and | | the presence of |
| Waste) | | soften the organics. | | steam for |
| | | Purpose: Treating | | subsequent waste |
| | | MSW in the presence | | separation and |
| | | of high temperature, | | recovery. |
| | | pressure and moisture | | This prior art needs |
| | | for the separation and | | external steam |
| | | recovery of inorganic | | generation. The |
| | | matter and organic | | subject invention |
| | | matter. | | uses steam |
| | | | | generated from the |
| | | | | wastes, so no |
| | | | | external energy is |
| | | | | needed for steam |
| | | | | supply. |
| U.S. Pat. No. 4,844,351 | Clifford | Method: Heat | No | This prior art needs |
| (Method for | Holloway | distortion and | | external steam |
| Separation, Recovery, | Jul. 4, 1989 | mechanical agitation | | generation. The |
| and Recycling of | | are used for plastic | | subject invention |
| Plastics from | | recycling. | | uses steam |
| Municipal Solid | | Process/Purpose: | | generated from the |
| Waste) | | After steam injection | | wastes, so no |
| | | and agitation by a | | external energy is |
| | | rotary kiln, rags is | | needed for steam |
| | | separated. Then | | supply. |
| | | magnetic and eddy | | After thermal- |
| | | current separators are | | chemical treatment |
| | | used to recover | | and plastic |
| | | ferrous metals and | | removal, the prior |
| | | aluminum. After that, | | art uses the |
| | | hot air is injected into | | remaining products |
| | | a trommel to separate | | for landfill or |
| | | plastics and organics | | incineration. |
| | | and other dense and | | However, the |
| | | rejects (such as bulk | | subject invention, |
| | | paper, glass bottles, | | after inorganic and |
| | | plastic bottles). By | | plastic removal, the |
| | | doing this to reduce | | remaining |
| | | the quantities of | | materials used for |
| | | wastes for landfill | | compost |
| | | disposal, and improve | | generation. |
| | | the quality of wastes | | |
| | | for incineration. | | |
| U.S. Pat. No. 4,908,098 | Edward A. | Method/Process: | Yes | This prior art uses |
| (Method for Extracting | Delong; | Lignocellulosic | | steam explosion to |
| the Chemical | Edward P. | materials are | | disassociate |
| Components from | Delong; | dissociated by steam | | lignocelluloses into |
| Dissociated | George S. | explosion. Then the | | celluloses, lignin, |
| Lignocellulosic | Ritchie; W. Alan | mixtures are extracted | | hemicelluses, and a |
| Material) | Rendall | by solvent extraction, | | mixtures of |
| | Mar. 13, 1990 | without the use of | | chemical |
| | | agitation. | | substances. Then |
| | | Purpose: | | water, alcohol and |
| | | Lignocellulosic | | caustic chemicals |
| | | materials are | | are used to extract/ |
| | | separated into water | | separate mixtures |
| | | soluble, alcohol- | | of chemicals. The |
| | | soluble, caustic | | subject invention |
| | | soluble, and cellulos. | | uses steam |
| | | | | explosion to |
| | | | | disassociate same |
| | | | | types of materials, |
| | | | | but used for |
| | | | | generation of |
| | | | | composts. |
| U.S. Pat. No. 5,190,226 | Clifford C. | Method/Process: A | No | This prior art |
| (Apparatus and | Holloway | rotatable pressure | | proposes a rotary |
| Method for | Mar. 2, 1993 | vessel, equipped with | | vessel and trommel |
| Separation, Recovery, | | extruder blades inside | | as the equipment |
| and Recycling | | the vessel is used to | | and uses steam |
| Municipal Solid Waste | | steam heating and | | without explosion |
| and the Like) | | agitation of the treated | | to separate wastes |
| | | solid wastes. A | | for recycling |
| | | trommel is used to | | materials such as |
| | | separate and recycle | | celluloses as fuel. |
| | | materials. | | The subject |
| | | Purpose: An | | invention use |
| | | apparatus is proposed | | steam explosion to |
| | | to recovery materials, | | disassociate |
| | | such as 50 to 65% of | | lignocelluloses to |
| | | recycled cellulose from | | produce more |
| | | MSW can be used as | | fibers and some |
| | | a fuel. | | humic substances |
| | | | | to be used as |
| | | | | composts. |
| | | | | This prior art needs |
| | | | | external steam |
| | | | | generation. The |
| | | | | subject invention |
| | | | | uses steam |
| | | | | generated from the |
| | | | | wastes, so no |
| | | | | external energy is |
| | | | | needed for steam |
| | | | | supply. |
| U.S. Pat. No. 5,262,003 | David E. | Method/Process/ | Yes | The prior art uses |
| (Method and System | Chupka; Peter | Purpose: A pressure | (explosion into a | steam explosion |
| for Defibering Paper | Seifert | digesting chamber is | liquid tank) | into water for fiber |
| Making Materials) | Nov. 16, 1993 | used for steam | | recovery to |
| | | treatment. Then the | | produce paper. |
| | | materials are | | The subject |
| | | discharged to a liquid | | invention uses |
| | | tank for fiber recovery | | steam explosion |
| | | to make paper | | to produce fiber |
| | | products. | | and humic |
| | | | | substances for |
| | | | | compost |
| | | | | generation. |
| | | | | This prior art |
| | | | | needs external |
| | | | | steam generation. |
| | | | | The subject |
| | | | | invention uses |
| | | | | steam generated |
| | | | | from the wastes, |
| | | | | so no external |
| | | | | energy is needed |
| | | | | for steam supply. |
| U.S. Pat. No. 5,361,994 | Clifford C. | Method/Process: A | No | This prior art |
| (Apparatus and | Holloway | rotatable pressure | | proposes a rotary |
| Method for | Nov. 8, 1994 | vessel, equipped with | | vessel and trommel |
| Preparation for | | extruder blades inside | | as the equipment |
| Separation, Recovery, | | the vessel is used to | | and uses steam |
| and Recycling of | | steam heating and | | without explosion |
| Municipal Solid Waste | | agitation of the treated | | to separate wastes |
| and the Like) | | solid wastes. Two | | for recycling |
| | | various sizes of the | | materials such as |
| | | conical blades are | | celluloses as fuel. |
| | | used concentrically, so | | The subject |
| | | waste entrance and | | invention use |
| | | exit are in the same | | steam explosion to |
| | | location. | | disassociate |
| | | Purpose: An | | lignocelluloses to |
| | | apparatus is proposed | | produce more |
| | | to steam treat wastes | | fibers and some |
| | | for separation and | | humic substances |
| | | recycling of materials. | | to be used as |
| | | | | composts. |
| | | | | This prior art needs |
| | | | | external steam |
| | | | | generation. The |
| | | | | subject invention |
| | | | | uses steam |
| | | | | generated from the |
| | | | | wastes, so no |
| | | | | external energy is |
| | | | | needed for steam |
| | | | | supply. |
| U.S. Pat. No. 5,556,445 | Mark K. Quinn | Method/Process/ | No | This prior art |
| (Steam Treatment of | Sep. 17, 1996 | Purpose: MSW is | | suggest a set of |
| Municipal Solid | | cooked, sterilized, | | apparatus to steam |
| Waste) | | soften and partially | | treat MSW at |
| | | hydrolyzed by the | | ambient pressure |
| | | rotating chamber | | for recycling. The |
| | | having an internal | | subject invention |
| | | perforated drum with | | use a partial |
| | | steam at ambient | | oxidation and |
| | | pressure to separate | | steam explosion |
| | | waste components | | processes to |
| | | and maintain moisture | | generate more |
| | | contents of products | | fiber and some |
| | | at 35 to 70%. | | humic substances |
| | | | | for compost |
| | | | | production. |
| | | | | This prior art needs |
| | | | | external steam |
| | | | | generation. The |
| | | | | subject invention |
| | | | | uses steam |
| | | | | generated from the |
| | | | | wastes, so no |
| | | | | external energy is |
| | | | | needed for steam |
| | | | | supply. |
| U.S. Pat. No. 5,618,003 | Frank M. | Method/process/ | No | This prior art use |
| (Process and | Akiyoshi; Lann E. | Purpose: Use | | steam treatment |
| Apparatus for | Richardson | pressurized steam | | without explosion |
| Reclaiming the | Apr. 8, 1997 | digester, mechanical | | to reclaim three |
| Components of Used | | chopper, and | | components |
| Disposable Sanitary | | screening process to | | (cellulose fiber, |
| Articles) | | disinfect and recycle | | absorbent granular |
| | | diapers. | | material, and |
| | | | | plastics) from the |
| | | | | disposable sanitary |
| | | | | articles. The |
| | | | | subject invention |
| | | | | use partial |
| | | | | oxidation and |
| | | | | steam explosion to |
| | | | | convert organic |
| | | | | wastes to |
| | | | | composts. |
| | | | | This prior art needs |
| | | | | external steam |
| | | | | generation. The |
| | | | | subject invention |
| | | | | uses steam |
| | | | | generated from the |
| | | | | wastes, so no |
| | | | | external energy is |
| | | | | needed for steam |
| | | | | supply. |
| US 2006/0225472 A1 | Helge Otto | Method/Process: | No | Due to no oxidant, |
| (Method of Converting | Friedrich Sahl | MSW is pretreated | | relatively low |
| Waste to Soil/Feed | Oct. 12, 2006 | with recyclable | | temperature, and |
| Modifiers) | | separation, radioactive | | short time period |
| | | detection, and grinding | | used by the prior |
| | | to 1 mm or less size. | | art, the putrescible |
| | | Then, the ground | | organics might not |
| | | waste is mixed with | | be completely |
| | | manure and sludge in | | decomposed to |
| | | a sealed drum. The | | form compost. |
| | | mixed contents is then | | It is known in the |
| | | sterilized by steam | | art that, as of the |
| | | with sufficient pressure | | current stage, due |
| | | at 120° C. for 37 | | to the complexity of |
| | | minutes, and cooled | | using enzyme for |
| | | for depressurizing and | | organic |
| | | venting the steam. | | decomposition and |
| | | Enzymatic solution is | | without cost- |
| | | added to facilitate | | effective methods |
| | | degradation. | | to generate |
| | | Purpose: MSW is | | significant amount |
| | | transformed to a | | of enzymes |
| | | useful compost | | commercially make |
| | | material. | | the “enzyme |
| | | | | treatment” still in |
| | | | | the R&D stage. |
| US 7,226,006 B2 | John A. | Method/Process: Hot | No | If the product |
| (Treatment of | Porter; Tony | water, shredding and a | | generated by the |
| Municipal Solid | Lees; | rotating drum, | | prior art is used as |
| Waste) | Paul A. Fitton | equipped with internal | | fuel there is no |
| | Jun. 5, 2007 | lifter blades., are used | | benefits to go |
| | | for treatment of MSW. | | through this |
| | | After that the MSW is | | process. If used |
| | | discharged into a | | for further compost |
| | | rotating thermal | | processing, the |
| | | processor. In which | | particle size (up to |
| | | the moist MSW is | | 8″) would be too |
| | | heated by heating the | | large for fiber |
| | | hot gases in the | | production. |
| | | processor by a flame. | | This prior art needs |
| | | which convert the | | external external |
| | | moisture in the MSW | | energy supply for |
| | | to steam. The steam | | processing. The |
| | | causes further pulping | | subject invention |
| | | of the MSW. Then, | | uses steam |
| | | the treated MSW is | | generated from the |
| | | transported to a | | wastes, so no |
| | | trommel screen for | | external energy is |
| | | separation of | | needed for |
| | | recyclables. | | cellulose |
| | | Purpose: Treatment of | | processing. |
| | | MSW into cellulosic | | |
| | | pulp for fuel or | | |
| | | composting use. | | |
| | | Other recyclable | | |
| | | materials reclaimed, | | |
| | | and inert material, | | |
| | | 10-15%, landfilled. | | |
| US 7,301,060 B2 | Brian S. | Method/Process: | No | This prior art uses |
| (Process for | Appel; | Subject the feedstocks | | thermal-chemical |
| Conversion of | James H. | to heat and pressure | | processes in a |
| Organic, Waste, or | Freiss; | in a reducing | | reducing |
| Low-Value Materials | William F. | environment | | environment to |
| into Useful Products) | Lange | accomplished by | | generate materials |
| | Nov. 27, 2007 | controlled addition of | | for further |
| | | sulfur and sodium, | | processing to fuels |
| | | separate out various | | and chemicals. |
| | | components, then | | The subject |
| | | further applies heat | | invention uses |
| | | and pressure to one or | | thermal-chemical |
| | | more of those | | processes in partial |
| | | components. | | oxidizing and |
| | | Purpose: Convert | | aqueous |
| | | organic wastes to gas, | | environment, and |
| | | oil, specialty | | subsequent anoxic |
| | | chemicals, and carbon | | environment for the |
| | | solids. | | production of good |
| | | | | quality composts. |

### SUMMARY OF THE INVENTION

1. Overall Treatment Processes and Objectives

The present invention addresses the processes and equipments for a high-rate production and improvement of the quality of composts from organic wastes. The overall process flow chart and objectives of the invention are summarizes in FIG. 1. As shown in FIG. 1, organic wastes, after Screening and Mechanical Pretreatment, are subject to three major treatment processes: Physical-Chemical Pretreatment, High-Rate Stabilization Process, and High-Rate Activation Process. The treated materials then go through Product Refining, and Bagging processes to generate finished composting products. Equipments used for the above mentioned screening, mechanical pretreatment, refining and bagging processes are commercially available. However, the three major treatment processes, i.e., Physical-Chemical Pretreatment, High-Rate Stabilization Process, and High-Rate Activation Process are developed by this invention. The operational sequences and chemical agents needed over various types of treatment processes listed in FIG. 1 are also developed by this invention. In order to ensure the one hour composting rate and high quality of compost products can be achieved, seven types of chemical agents may be required. These seven types of chemical agents are: Wetting Agents, Debonding Agents, Organic Stabilization Agents, Inorganic Stabilization Agents, Fluffing Agents, Activation Agents, and Nutrients (N, P and K fertilizers) Agents.

2. Waste Screening Process

Incoming wastes for the subject treatment and recycling are first going through the Screening Process. There are three types of objectives/operations involved in the Screening Process: (1) Separation and recycling of inorganic materials in the incoming wastes, (2) Separation and recycling of improper organics (mainly refractory organics such as plastics, rubber and synthetic fabrics in the wastes), and (3) selection and purification of the incoming wastes. Not all the incoming wastes require to process through the above mentioned operations. In general, only MSW and wastes from landfill mining (cleanup) need to process for the above listed objectives. Selection and purification of wastes mentioned above can be designed based on organic types desired, nutrient contents, and special types of compost to be generated. For example, when municipal wastewater treatment plant sludge is processed, in order to generate higher quality of composts, wastes containing higher fibrous materials such as municipal wastes, green wastes or agricultural wastes can be added.

Waste Screening Process can be practiced partly manually and partly by machine, or totally by machine depending on incoming waste characteristics, and cost-effectiveness. If without manual processing, this process can also be done after Mechanical Pretreatment, or after Physical-Chemical Pretreatment processes. As shown by FIG. 2, for MSW recycling, it is suggested that some types of manual processing is involved for garbage bag breaking, and recovery of entire objects (without size reduction) of recyclables such as plastic bottles, metallic cans, glass containers, plastic bags, used films and CD/DVD's, fabrics. By processing this way, subsequent recovery by delivery to various types of recycling plants can be facilitated. Waste Screening Process shall be operated indoor with negative pressure to avoid odor dissipation problems. Air extracted from the building shall be treated by processes such as activated carbon, scrubbing, and other air purification methods. After the recycling activities, the incoming wastes can further processed by other commercially available solid waste separation and purification machines such as vibrating screens, trommels, disc screens, etc. for inorganic removal, when necessary.

3. Mechanical Pretreatment Process

As shown in FIG. 1, Mechanical Pretreatment involves three types of objectives/operations: (1) size reduction, breaking and disassociating of fibrous materials, (2) further purification and removal of impurities, and (3) mixing of different incoming waste materials. Therefore, the ultimate objective of this pretreatment process is to prepare a raw material with high purity and desirable ingredients for high quality composts production and preconditioning of the raw materials suitable for processing in the following three major processes invented by the subject invention.

In this pretreatment process, one of the most important operation is to pre-treat the lignocellulosic materials. It is known in the art that higher adsorption and absorption characteristics of composts can be achieved when more fine and disassociated cellulosic fibers can be obtained from lignocelluloses. Advantages of this operation and the subsequent physical-chemical pretreatment, stabilization, and activation operations developed by this invention are the essential parts of the invention. These operations and equipments suggested by this invention can achieve good quality of composts which traditional composting processes can hardly or will never achieve. This type of improvement is also not emphasized by other existing composting processes, such as hydrolysis processes.

Lignocelluloses are most abundant and valuable organic materials which are produced by plants on earth. Based on data published by USEPA for MSW in 2006, contents of lignocelluloses and their disassociated materials are calculated over 50% in average MSW in USA. After inorganic recovery and refractory organics removal as discussed above, contents of lignocelluloses in the processed MSW can be as high as 70 to 80% (wet basis). Celluloses disassociated from lignocelluloses are the major ingredients for the good quality compost production. Cellulosic fibers and their derived humic substances have high nutrients adsorption and water absorption capacities. Through addition of wetting and debonding agents, fibrous materials can be further modified to enhance the above characteristics and other improvement for soil physical structure modification. Lignocelluloses can be disassociated and decomposed through methods such as white fungus decomposition, enzyme hydrolysis, dilute acid hydrolysis, concentrated acid decomposition, other chemical decomposition (such as adding sodium hydroxide, sulfur dioxide, liquid nitrogen, phosphorous acid, alkaline hydrogen peroxide, ammonium salts, acetic acid, etc.), physical decomposition (such as microwave irradiation), and high temperature high pressure steam decomposition. However, no matter which method is used, pretreatment of lignocelluloses by size reduction and disassociation of fibers are necessary steps to facilitate the reaction. The subject Mechanical Pretreatment Process is, therefore, designed.

This Mechanical Pretreatment Process involves shredding and grinding operations. The following commercially available equipments are available for the subject purposes: hammermills, grinders, and shredders. Sizes to be reduced and degree of fiber disassociations needed for compost production shall be selected based on different application requirements, such as for desertification control, this invention suggests that sizes can be larger (in general, it can be in the range of 0.1 mm to 1 cm or larger, near the sizes of fine sands to fine gravel classified by USDA), due to requirements for wind and water erosion control and replacement for the lost fine soil particles in deserts. For crop fields or landscaping soil improvements, sizes to be mechanically pretreated can be smaller, such as in the range of 0.05 to 2 mm. If used for soil surface cover to prevent weeds growing or protect germination, sizes can be as large as 1 cm to 5 cm.

The second objective of Mechanical Pretreatment Process in this invention is for further purification of incoming raw materials. After size reduction to more uniform sizes, organics and inorganics in the incoming wastes can be separated more cost-effectively by densities such as using the following equipments: air classifiers, inertial separation, and air knife classifier. Inorganic portion separated can be further separated for further recycling, such as unit operations listed as “f, g, h” in FIG. 2.

In certain cases the subject Mechanical Pretreatment Process also provides for pre-processing and mixing of raw materials. Suitable wetting and/or debonding agents can also be added right before size reduction operation to enhance disassociation of lignocelluloses and saving energy for size reduction operation.

4. Utilization of Natural and/or Artificial Chemical Agents

This invention presents seven types of chemical agents for the purposes of enhancing quality of composts, and removal of toxic compounds when necessary, they are: Wetting Agents, Debonding Agents, Organic Stabilization Agents, Inorganic Stabilization Agents, Fluffing Agents, Activation Agents, and Nutrients (N, P and K fertilizers) Agents. Necessity of applying any of these seven types of agents depends on types of raw organic materials to be processed, operation methods selected, higher degree of product quality anticipated, and types of compost application planned. For example, if the compost products are used for organic food production, then agents from all-natural sources shall be selected. Due to wide varieties of agents available, knowledge and experience on chemical agents usage become important for the agents selection. Efforts also shall be made to select an agent which can achieve more than one purposes, such as certain Wetting Agents can also be used as Activation, Fluffing and Nutrient agents. Purposes, examples of chemicals available, and dosage needed are further discussed by this invention below.

### Wetting Agents:

In the production of composts the basic advantage of adding Wetting Agent(s) is to infiltrate into the surface of fibrous materials to disassociate fibers which are negatively charged and with strong hydrogen bonding holding fibrous material together. Depending on types of Wetting Agents and needs of adding Wetting Agents, the following advantages can also be achieved:
(1) Some wetting agents have the wet expansion or heat expansion characteristics which can further loosening fibers in the succeeding operations when water or heat is added;
(2) Certain wetting agents when added prior to the mechanical shredding/grinding operations can save energy consumption by lubricating effects;
(3) Some wetting agents also are strong adsorbents and/or strong absorbents or containing nutrient ingredients;
(4) Certain wetting agents can assist other fluffing agents (to be discussed latter) to infiltrate into fibers;
(5) Composts with certain wetting agents can assist soil to reduce its water surface tension and increase soil's water affinity and transmissibility.

The subject invention presents two types of wetting agents for compost production: organic and inorganic types. Inorganic wetting agents presented by this invention include expansible clay minerals (such as montmorillonite, especially sodium montmorillonite, or called bentonite, and kaolinite, vermiculite, perlite, etc.) and multi-valenced and positively ionized metallic compounds which can infiltrate into negatively charged fibers (such as alum or aluminum sulfate, titanium dioxide, etc.). The inorganic wetting agents can be used for the subject invention also include chemical compounds which can assist expansion and softening of fibers, such as carbonates (sodium carbonate, magnesium carbonate, calcium carbonate, ammonium carbonate, etc.), and bicarbonates (sodium bicarbonate, ammonium bicarbonate, etc.). Organic wetting agents can be used for the subject composting process include various types of fatty acid esters, and non-ionic surfactants. Examples of fatty acid esters are glycerol monostearate, glycerol monooleate, diethylene glycol monostearate, diethylene glycol monooleate, propylene glycol monooleate, etc. Among them fatty acids of alcohols containing at least one ether group are more suitable to use for the subject purpose, such as diethylene glycol, triethylene glycol, polyethylene glycol, and polypropylene glycol. Non-ionic surfactants can be used for the subject composting as wetting agents are commercially available such as Triton X-100, Triton X-45, Triton X-114, etc. Suitable dosage range for the above inorganic or organic wetting agents is from 0.5 to 5% (based on dry weight of celluloses in raw materials for composting). The most suitable dosage for most composting conditions is about 1% wetting agent (based on dry weight of celluloses).

### Debonding Agents:

Mechanical Pretreatment for compost production usually reduces the sizes of ligcelluloses to mm dimensions. In general, elementary fibrils of celluloses are in the nanometer-sizes, about 30 nm×3.5 nm. Cellulosic microfibrils are embedded in a matrix of hemicellulose and protolignin at sizes around 25 nm×30 nm. Therefore, in order to further enhance adsorption, absorption and chelating effects of celluloses Debonding Agents can be used to expose more cellulosic microfibrils. In this invention the cationic quaternary ammonium compounds used widely in the paper industry also can be selected as Debonding Agents for compost production. These types of compounds can penetrate into negatively charged celluloses relatively easy. Examples of these types of compound include trimethylalkyl ammonium halides, trimethylalkylene ammonium halides, methylpolyoxyethylene alkylene ammonium halides, etc. as shown in the following common formula:

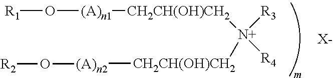

Where: R₁ and R₂=aliphatic hydrocarbons with 12 to 40 carbons;
R₃ and R₄=methyl, ethyl, hydroxyethyl groups;
A=oxyalkylene group, derived from both ethylene oxide and propylene oxide;
m=a number corresponding to the valence of X;
ₙ₁ and ₙ₂=average number of oxyalkylene units (6 to 30);
X=anion.
   However, celluloses will reduce their water absorption capacities and loosing strengths when cationic quaternary ammonium compounds were used. Therefore, addition of anionic or non-ionic agents (can be Wetting Agents as discussed above or Fluffy Agents to be discussed latter) into composts after debonding processing may, be needed. Another type of problem of using cationic quaternary ammonium compounds is their toxicity and skin irritative effects. This invention will avoid using these compounds unless they are used for non-food related composts. The cationic quaternary ammonium halides are also corrosive to steel reactors. As a result, this invention suggests to use more suitable Debonding Agents such as mixtures of a phospholipids, a non-ionic surfactant, and optionally a lubricating additive, also used as Fluffing Agents discussed latter. Dosage amount for the Debonding Agents fall in the range of 0.5 to 5%, but 1% of dosage shall be sufficient for compost production.

### Organic Stabilization Agents:

The traditional composting methods apply microorganisms to stabilize/decompose easily decomposable organics by using enzyme as catalyst to pull oxygen and organic together for oxidation. This invention is using high temperature and pressure to enhance the oxidation of organics. Oxidants presented by this invention for composting include ozone, chlorine, hypochlorites, potassium permanganate, hydrogen peroxide, oxygen, etc. In order to save costs and simplify operation, partial wet oxidation by pressurized air is a more suitable method. Since reactions occur in high temperatures, in theory, reaction rates will be doubled when temperature increases in 10 degree. Therefore, theoretically, when reaction temperature increase from 20° C. to 200° C. the reaction rate would be 2¹⁸ times (or 262,144 times) increased. In this situation, if composting reaction happened at 60° C. for two months (such as optimum traditional composting processes), the reaction time period can be reduced to about 5 minutes if 200° C. is used. The one hour high rate composting can be achieved is partly based on this principle. This invention also uses high pressure which can dissolve more organics and oxidants into the solution, therefore, further enhance the oxidation rates. Under these conditions, when temperature and pressure are suitable, some of moderately decomposable toxic compounds such as PCB's, dioxine, benzene, PAH's, pesticides, and insecticides can be significantly decomposed by the above listed oxidants.

### Inorganic Stabilization Agents:

If the inorganic toxic compounds, such as heavy metal compounds, existed in composts beyond the legal limits, Inorganic Stabilization Agents can be used to overcome the problem. Certain adsorption, chelating or ion-exchange effects were presented by prior arts (such as CN101172899, 2008; CN101274861, 2008; and CN101322973, 2008) for heavy metal stabilization in composts. However, when these types of treated composts are applied to agricultural fields, crop roots could still uptake the stabilized heavy metals, due to the reversible characteristics of the adsorption, chelating, and ion-exchange effects.

Heavy metals in wastes (such as MSW, municipal treatment plant sludge) or in natural environments (such as soils, sediments) are existed in many different forms (such as dissolved, solid) or chemical species (such as chelated, reducible compounds, complicated minerals). Some of the species are very stable and will not release out for uptake by plant roots, such as metals incorporated into silicate crystals, or form stable and least soluble solid species. In wastes or soils, the exchangeable, adsorbed, or chelated metal species are usually staying on the surface of the solid particles and are relatively mobile for release into the soluble phases, which are available for roots to uptake. In order to explain metal stabilization methods presented by the subject invention, it is necessary to discuss possible chemical species existing in wastes and soil. In general, heavy metals can exist in wastes, soils or sediments in the following five categories:
(1) Water soluble species;
(2) Exchangeable, adsorbed or chelated species;
(3) Reducible species;
(4) Oxidizable species; and
(5) Lithogenous species.
   All of above five categories of metal species can co-exist in wastes and soils. Stability of metal species is increased from (1) to (5), as listed above. When metals are existed below category (2) as listed above, metals become relatively unavailable to vegetations. Metals usually form least soluble solid species under the environments which they are staying. Metals which are available to vegetations under this situation only through dissolution effects of the least soluble solid species, which is usually very slow and therefore impacts to vegetation would be minimal. The least soluble metal solid species for any given metal can be gradually formed depending on reducing or oxidizing environments, as shown below:

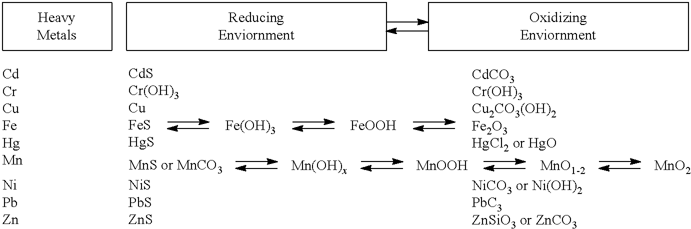

Two approaches are presented by the subject invention for the control of heavy metals in composts: (1) extraction method, and (2) transformation method. Method (1) uses appropriate chemicals to extract heavy metals from the wastes. Method (2) provides chemicals and suitable environments to convert metal species to the least soluble metal solid species. Both methods can be used together, or individually. Selection of the method will be based on type and concentration of metals in the incoming wastes, regulatory requirements, future intended use of the compost, and future possible soil conditions for compost application (e. g., pH, redox, and existing metal concentrations). For example, if extraction method can remove easily the metal concentration below regulatory requirements, then the transformation method will not be required. However, if easily releasable metals (i.e., exchangeable, adsorbed or chelated metals) are abundant in compost after extraction process, in order to improve compost quality, transformation method may be used.

The extraction method can remove easily releasable metals from composts by leaching out the exchangeable, adsorbed and chelated metal species with appropriate chemicals. This invention presents the following chemicals for the extraction of metal species from wastes for compost production:
Chemicals for extraction of exchangeable, adsorbed and chelated metals: NH₄Ac, Ca(NO₃)₂, Mg(NO₃)₂, MgCl₂, NH₄Ac+NH₄OH (pH=9), 2% citric acid, 0.1N HCl, 0.2M ammonium oxalate, and EDTA.
Chemicals for the extraction of reducible metal species: 1N NH₄Ac+0.2% hydroquinone, NH₂OH.HCl, 0.04M to1M NH₂OH.HCl+25% HAc, sodium dithionite-sodium citrate, dilute acids, and NH₂OH.HCl+dilute acids.
Chemicals for extraction of oxidizable metal species: H₂O₂, sodium hypochlorite, H₂O₂+dilute acids, ozone, chlorine, other hypochlorite salts, potassium permanganate, oxygen, and the above listed oxidants plus dilute acids.
Chemicals for extraction of lithogenous metal species: strong acids, and mixtures of strong acids, especially HNO₃+HF+HClO₄.
   Extraction power of the above mentioned chemicals increases from top category (i.e., category for exchangeable, adsorbed and chelated) to lower metal categories for metal extraction. Therefore, chemical selected for the lower metal category will extract metal species in the higher metal categories also. For example, when choose a dilute acids for reducible metal extraction, the exchangeable, adsorbed and chelated metal species also will be removed. If extraction method is used for compost production operation, extractant shall be separated from the treated waste materials so metals can be removed from the wastes. However, after treatment of the extractant, it should be recycled back to the composting processes to avoid loosing of any valuable nutrient contents. This practice also can achieve zero discharge objective for wastewater generated. Extractants can be treated by conventional metal removal processes such as carbon adsorption, ion-exchange, membrane separation (e.g., reverse osmosis, electrodialysis, ultrafiltration), chemical precipitation, etc.

In employing the Metal transformation method, this invention suggests to transform metal species to the most stable, or least soluble solid species in the oxidizing environments. As shown above, examples of these types of solid are CdCO₃ for Cd, Cr(OH)₃ for Cr, ZnSiO₃ or ZnCO₃ for Zn, etc. Oxygen (or air) can be used to maintain DO value in the reactor to beyond 2 ppm in the solution during transformation processing. Chemical compounds which can generate related anions to form the anticipated least soluble solid species shall be added into the reactor to assist the reducible solid formation. For example, for CdCO₃ solid formation carbonate salts can be added, for Cr(OH)₃ solid formation hydroxides to raise pH can be added, for ZnSiO₃ solid formation soluble silicates can be added, etc.

### Fluffing Agents:

Debonding Agents can assist to loosening cellulosic fibers but some agents could also reduce the water absorption capacity and fiber strength of celluloses. In these situations, Fluffing Agents shall be added to recover the water absorption capacity and fiber strength of celluloses. Fluffing Agents also can be separately used to further enhance compost quality in water absorption, air circulation, and thermal insulation characteristics.

This invention presents three types of Fluffing Agents for achieving the above stated compost quality improvements:
(1) Add cation retention agents and anionic or non-ionic surfactants to dry incoming wastes right before size reduction operation (i.e., right before the Mechanical Pretreatment), or after cellulosic pulp formation stage (i.e., the final stage of the High-Rate Stabilization Process, to be discussed latter).
(2) Add mixtures of phospholipids, a non-ionic surfactant, and an optionally a lubricating additive right before size reduction operation or after High-Rate Stabilization Process.
(3) Add an antioxidant and hydrophilic agent, as well as an inorganic swelling chemical right before the final stage of the steam explosion operation (i.e., High-Rate Activation Process, to be discuss latter).

Chemicals for the type (1) method listed above include aluminum sulfate (as a cation retention agent) plus paraffin (as a non-ionic surfactant), or cationic quaternary ammonium compounds plus nonionic fatty acid esters. Phospholipids in the type (2) method include phosphatidylcholine or lecithin, hydroxylated phosphatidylcholine, phosphatidylethanolamine, etc. Non-ionic surfactants mentioned in the method (2) above are similar to that used for Wetting Agents such as diethylene glycol, triethylene glycol, polyethylene glycol, and polypropylene glycol and Triton X-100, Triton X-45, Triton X-114, Igepal CO-630, Igepal CO-430, etc. Plant oils such as olive oil, caster oil and other vegetation oils are candidates for lubricating additives. Method (3) are mostly inorganic compounds such as Na₂SO₃, K₂SO₃, MgSO₃, and (NH₄)₂SO₃ for antioxidant and hydrophilic agents, and MgCl₂, Na₂CO₃, NaHCO₃, (NH₄)₂CO₃, MgCO₃, and NH₄HCO₃ for swelling chemicals. This invention presents the dosage amount of all the above listed chemicals at 0.5 to 5% (dry weight basis of celluloses in the incoming wastes). Among the above chemical candidates, this invention suggests that Method (3), especially those chemicals also have nutrient ingredients shall be priority candidates due to the reason that, besides the advantages of adding Fluffy Agents, the compost nutrient contents can be increased and many of the least soluble metal solid species (such as carbonates of Cd, Cu, Ni, Pb, and Zn) can be also formed.

### Activation Agents:

Objectives of Activation Agents are to enhance compost's nutrients and essential micro elements adsorption/exchange capacities and water absorption capacity. The ultimate effects are to increase the plant growth rates and reduce potential for water pollution. This invention presents two approaches for activation of compost products: (1) improvements of cellulosic and humic substances, (2) addition of Activation Agents.

As discussed previously, the best compost activation agents would be the cellulosic and humic substances. Mechanical grinding, biological decomposition (such as decomposed by white fungus), chemical treatment (enzyme hydrolysis, dilute acid hydrolysis, strong acid decomposition, and decomposition by other chemical compounds such as sodium hydroxide, sulfur dioxide, liquid ammonia, phosphorus acid, alkaline hydrogen peroxide, ammonium salts, acetic acid, and formic acid), physical decomposition (such as microwave irradiation), and steam explosion can all achieve certain degrees of compost activation. Some of the Wetting, Fluffing, and Debonding Agents discussed previously also can achieve the objectives.

However, in order to further improve compost quality, Activation Agents can be added. It is critical that agents with high activation effectiveness, non-toxicity, low costs, and availability (such as for organic food production, activation agents shall be all natural products) shall be selected. This invention presents two types of Activation Agents can be added into the final stages (such as in the final stage of the High-Rate Activation Process, or in the product Refining Process, as shown in FIG. 1) of compost production: (1) Inorganic Activation Agents, such as clay minerals (bentonite, kaolinite, vermiculite, perlite, zeolite, etc.), and activated carbon. (2) Organic Activation Agents, such as peat, brown coal. Dosage of Activation Agents for compost production can be in the range of 1 to 50%, depending on anticipated capacities of adsorption and absorption to be reached.

5. Physical-Chemical Pretreatment

As shown in FIG. 1, major objectives of the Physical-Chemical Pretreatment are:
Pretreatment for lignocelluloses;
Pretreatment for cellulose debonding.
   This pretreatment operation includes Wetting Agent and/or Debonding Agent addition, mixing, preheating, and moisture preconditioning. For co-treatment involving sludge or slurry types of input wastes, wastes can be input into this operation process directly due to no mechanical size reduction is needed. Mixing is provided for waste/waste mixing and wastes/chemical mixing. Waste heat (steam) generated by other downstream operation processes can be recycled to this pretreatment tank to preheat the waste material. This operation tank is also used to adjust the moisture contents of the mixed wastes in order to meet the requirement for the next operation process, i.e., High-Rate Stabilization Process.

6. High-Rate Stabilization Process

Seven major objectives are involved in this compost production process:
Stabilization of easily biodegradable organics and nutrient release;
Stabilization/removal of heavy metals, when needed;
Preliminary separation/disassociation of lignocelluloses;
Pathogens and parasites sterilization;
Detoxification;
Provision of operation energy; and
Improvement of compost qualities.

### High-Rate Stabilization of Easily Decomposable Organics:

The first item listed above is one of the most important objectives listed above—decomposition or stabilization of easily biodegradable organics in the input wastes. This objective alone is equivalent to the overall objective of the traditional composting process. Oxidant(s) are used in this process to decompose easily decomposable organics in about 20 to 30 minutes under moderate temperature and pressure ranges. Oxidants suggested by this invention for the subject objective includes any or combinations of the following: ozone, oxygen, hypochlorides, potassium permanganate, and hydrogen peroxide. Air can also be used to supply oxygen.

In order to achieve the high-rate reactions, reactors used shall meet the following conditions:
Suitable oxidant(s) concentrations and temperatures must be maintained in order to nearly complete oxidation of the easily decomposable organics, while formed cellulosic fibers and humic substances are relatively intact;
Suitable pressures must be maintained in order to keep water and oxidant(s) in the liquid phase;
If heavy metal removal is necessary, chemicals selected shall be able to achieve multiple purposes such as metal extraction down to legal limits, assistance in decomposition of easily biodegradable organics, assistance in fiber and humic substances formation, and assistance in providing nutrient contents; and
Maintain short reaction time period (usually less than 30 minutes) for achieving the above objectives.
   Regarding to the suitable oxidant concentrations mentioned above, this invention provides a rough estimate method: 1O₂=1C, where “C” is the molar amount of carbon in the easily decomposable organics. “O₂” is the molar oxygen equivalent amount. For example, in MSW the easily decomposable organics are related to food wastes, which is about 6 to 26% in MSW. In the average USA MSW in 2006, this portion is about 12.4%. The oxygen demand also can be estimated through BOD and COD tests. For example, in municipal sludge the BOD values are close to the oxygen demand by the easily decomposable organics, and the BOD amount is near 25 to 35% of COD values. Since COD can be analyzed faster, it can be used as an indicator for the status of decomposition of easily decomposable organics in the subject reactor. The concentration of oxidants in the reactor is in the range of 1 to 6 ppm of oxygen equivalent.

As discussed previously in this invention, the temperature, pressure and reaction time period ranges suitable for the subject High-Rate Stabilization reactor are usually lower than that for complete wet-air oxidation process. Based on the subject invention for the compost production, the following criteria can be used for the temperature, pressure, and time period selection:
Temperature Range: The minimum temperatures used shall be higher than the self-sustaining reaction temperature. For most waste types, this minimum self-sustaining temperature is close to 140° C. However, the maximum temperature is usually smaller than 250° C., depending on types of wastes to be treated. The best temperature range is between 180° C. and 230° C. In certain special cases such as detoxification of toxic organics, sterilization of pathogens and parasites, decomposition of pesticides, etc. the temperature requirements will be higher, such as in the 250° to 300° C. range. The above temperature requirements can be reduced when the reaction time is increased. When the metal extraction chemicals are used, the temperature requirements can also be reduced.
Pressure Range: Pressure requirements are related to temperature selected. In general, for the subject stabilization purpose, pressure shall be higher than that formed by the saturated water vapor pressure at the selected temperature. In order to increase dissolved oxidant and organic concentrations, minimum pressure requirement for the subject composting stabilization is 50 to 100 psi (approximately 3.5 to 7 atm) higher than the corresponding saturated water vapor pressure generated by the temperature in the reactor. The better temperature and pressure ranges for the subject invention are shown in the following Table 1.
Reaction Time: Reaction time needed is reverse proportional to temperature and chemicals added for the metal removal. The reaction time needed is also affected by the types of inputting wastes. The best time needed can be done through bench tests based on the above mentioned parameters. In general, for the subject composting, 5 to 20 minutes are usually sufficient for organic stabilization. Lengthy time period could increase hydrolysis and oxidation reactions on celluloses and reduce the compost amount generated.

TABLE 1
Temperature and Pressure Requirements for the
Stabilization of Easily Decomposable Organics

| | |
|---|---|
| | Minimum Pressure |
| Temperature | Requirement |

| (° C.) | psi | atm |
|---|---|---|
| 140 | 102 | 7 |
| 145 | 110 | 7.5 |
| 150 | 119 | 8 |
| 155 | 129 | 8.8 |
| 160 | 140 | 9.50 |
| 165 | 152 | 10 |
| 170 | 165 | 11 |
| 175 | 179 | 12 |
| 180 | 195 | 13 |
| 185 | 213 | 14.5 |
| 190 | 232 | 15.8 |
| 195 | 253 | 17 |
| 200 | 276 | 18.7 |
| 205 | 300 | 20.4 |
| 210 | 327 | 22 |
| 215 | 355 | 24 |
| 220 | 386 | 26 |
| 225 | 420 | 28.6 |
| 230 | 456 | 31 |
| 235 | 494 | 33.6 |
| 240 | 535 | 36.4 |
| 245 | 580 | 39.4 |
| 250 | 627 | 42.6 |

The High-Rate Stabilization Process is an exothermic reaction. For most easily decomposable organics if the temperature selected is greater than 140° C. and easily decomposable organic contents are higher than 5%, and heat contents of the organics are greater than 3,000 Btu/pound, then no external energy supply is needed for the temperature and pressure maintenance. The steam generated through this process also enough for the supply to other operation processes which require steam such the Physical-Chemical Pretreatment, High-Rate Activation Process, and Product Refining process. In this way a great saving can be achieved by using this invention for the compost production.

### High-Rate Stabilization of Heavy Metals:

If heavy metal contents are exceeding legal limits of composts, the same reactor also can be provided for the metal extraction and transformation reactions. Chemicals discussed above can be selected depending on various conditions as discussed previously. Due to high temperature and oxidation environments used for the High-Rate Stabilization Process, reducing agents, or unstable chemicals cannot be used. In this invention, dilute acids maintaining the reactor pH close to or less than 4 will be sufficient for most heavy metals (such as Cd, Cr, Cu, Fe, Hg, Mn, Ni, Pb and Zn) removal. Dilute acids can also assist in cellulose fiber dissociation and humic substances production. If acids are used, neutralization shall be applied to the treated material right after dewatering for the metal removal.

If multiple wastes are involved for the compost production and only some of the wastes containing metals exceeding limits, they can be treated before the stabilization process such as in the Physical-Chemical Process, or in a separated reactor. In this case the temperature is lower and environmental conditions can be also individually adjusted to fit more favorable chemicals to be used for extraction or transformation.

### Other High-Rate Stabilization Functions:

Other objectives as listed above can also be achieved through the High-Rate Stabilization Process, such as through hydrolysis and oxidation resulting in disassociation of lignocelluloses; through the use of high temperature causing pathogens and parasites sterilization; through strong reaction capability caused by high temperature, high pressure, high contact opportunities among organics and oxidants destructing toxic organic compounds; through energy (steam) generation from spontaneous oxidation reactions providing operation energy internally; etc.

In order to achieve all of the above objectives, one of the most critical criteria is to keep oxidant(s), additives and organics in a dissolved forms and promoting high rate contact opportunity for reactions. Therefore, the types of reactor design become very important. Both vertical and horizontal reactors can be used. The key is to uniformly and quickly dissolving oxidant(s) throughout the input mass of waste materials. Short-circuiting effects of the reactor shall be control to a minimum. The subject invention presents a design example latter in this document to illustrate the above principles.

7. High-Rate Activation Process

In this invention Fluffing Agents, Activation Agents, and saturation steam are used to treat the stabilized materials under high pressure environments. Major materials existed under this condition are disassociated fibrous cellulosic and humic substances. Objectives of this operation process are:
High rate disassociation of lignocelluloses to form fibrous cellulosic and humic substances;
Further enhancement of nutrients adsorption and water absorption capacities, and water and air circulating and thermal insulation characteristics;
Further pathogen and parasite sterilization;
Further detoxification; and
Further improvement of compost activation and stabilization characteristics.

This invention presents the following treatment conditions for achieving the above objectives:
Temperature Range: In order to save energy, steam and temperature formed after the High-Rate Stabilization and subsequent operation processes are used. The temperature range is usually between 140° and 250° C., preferably 180° and 230° C.
Pressure Range: Pressure needed is the saturated water vapor pressure formed at the temperature used. This can be obtained from the pressure used in the High-Rate Stabilization Process minus about 50 to 100 psi (about 3.5 to 7 atm) pressure. Table 2 presents a more favorable pressure range suggested by this invention for compost production.

TABLE 2
Temperature and Pressure Requirements for the
Activation of Fibrous Cellulosic and Humic Substances

| | |
|---|---|
| | Pressure |
| Temperature | Requirement |

| (° C.) | psi | atm |
|---|---|---|
| 140 | 52 | 3.5 |
| 145 | 60 | 4.1 |
| 150 | 69 | 4.7 |
| 155 | 79 | 5.4 |
| 160 | 90 | 6.1 |
| 165 | 102 | 6.9 |
| 170 | 115 | 7.8 |
| 175 | 129 | 8.8 |
| 180 | 145 | 9.9 |
| 185 | 163 | 11.1 |
| 190 | 182 | 12.4 |
| 195 | 203 | 13.8 |
| 200 | 226 | 15.4 |
| 205 | 250 | 17 |
| 210 | 277 | 18.8 |
| 215 | 305 | 20.7 |
| 220 | 336 | 22.9 |
| 225 | 370 | 25.2 |
| 230 | 406 | 27.6 |
| 235 | 444 | 30.2 |
| 240 | 485 | 33 |
| 245 | 530 | 36.1 |
| 250 | 577 | 39.3 |

Reaction Time: This invention discovered that 5 to 20 minutes are sufficient to achieve the above stated objectives. If reaction time is too long the hydrolysis and oxidation could further decompose cellulosic and humic substances, and therefore reduce the amount of compost produced. The reaction time is reverse proportional to the temperature selected, as can be shown by the following empirical formula:

Reaction Time=(1,500 to 3,000 minutes·temperature)/Temperature
Moisture Contents: Best moisture contents of treated material in the High-Rate Activation reactor can be maintained at the Field Capacity of the treated materials.

Requirements for the use of Fluffing and Activation Agents will be based on the needs for compost quality. Dosage ranges are presented previously in this invention. After the high pressure steam treatment, steam explosion is used to further disassociate fibrous materials. In order to maintain enough pressure for steam explosion, minimum 3 atm pressure difference shall be selected for better results. Reactors such as auger digesters, rotary kilns, and autoclaves can be used.

8. Product Refining

As shown in FIG. 1, three objectives are involved for product refining:
(1) Moisture control;
(2) Particle size control; and
(3) Product nutrient control.
   Moisture is usually controlled to less than 35% (weight basis), or based on any requirements from regulatory requirements for compost products. Commercially available dryers or centrifuges can be used to achieve the objective. Compost product particles are usually controlled below 12 mm sizes. However, depending on special needs for application, as discussed previously, sizes can be adjusted. Nutrient contents in the composts produced from most waste materials are usually less than 1% for N, P and K. Based on market needs or for the purpose of increasing product values, NPK chemical compounds can be added. This objective can be achieved under this product refining process.

### BRIEF DESCRIPTION OF THE DRAWINGS

The invention will be described in more detail by way of example only, with reference to the accompanying drawings. The following drawings are provided:

FIG. 1 shows the BFD (Block Flow Diagram) with unit operation processes and objectives of each process of the subject invention. It also shows the most appropriate locations for chemical additives.

FIG. 2 shows overall flow charts of the compost production systems for different types of organic wastes.

FIG. 3 shows a typical P&ID (Processes and Instrumental Diagrams) of a composting plant using the subject invention.

FIG. 4 shows an example of profiles of the major reactors of the subject invention.

### DETAILED DESCRIPTION OF THE PREFERRED EMBODIMENTS

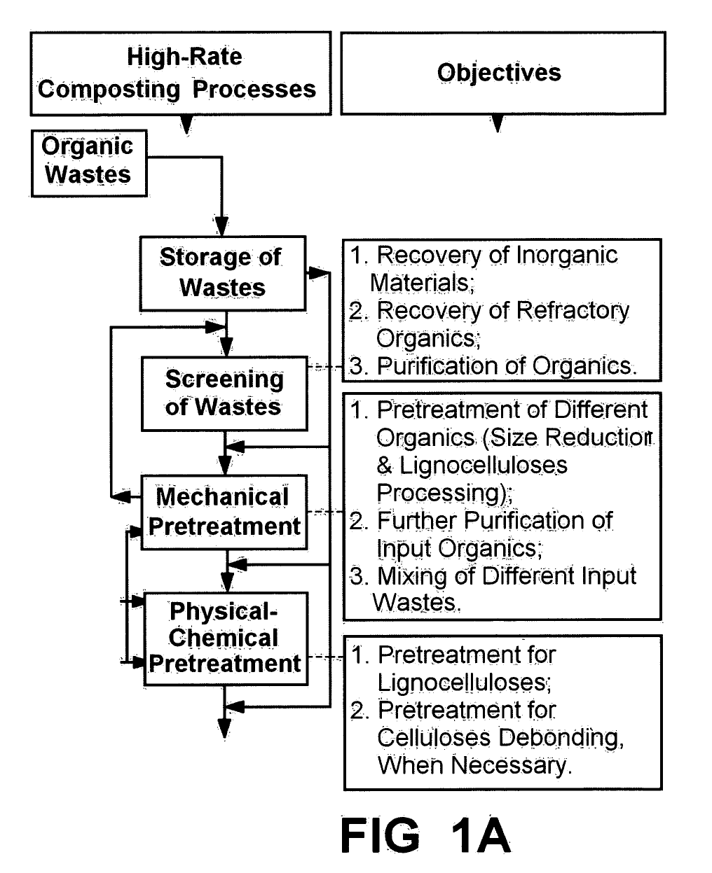

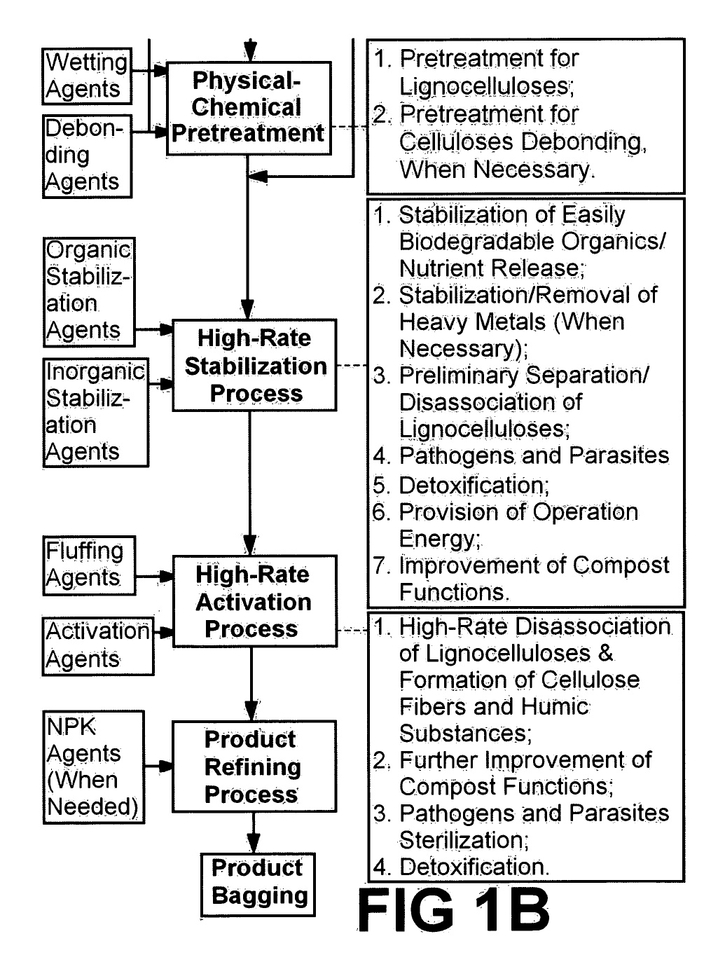

Referring to the drawings, FIG. 1 shows the BFD (Block Flow Diagrams) of the subject processes, objectives of each unit process, and application locations and types of chemical agents to be applied in this subject invention. As shown in FIG. 1, Storage Facilities are provided for receiving organic wastes to be treated. The Storage Facilities shall be closed tanks if odor is a problem. If closed tanks can not be used, the Storage Facilities shall be in indoor designed with negative pressure to prevent odor problem. After the Storage Facilities, Screening Processes are provided for separation and purification of wastes to remove unsuitable or undesirable materials from the incoming wastes such as inorganic materials and refractory organics. In general, MSW is the only type of wastes which require these processing. The next process, Mechanical Pretreatment, is provided for further purification of incoming wastes and for size reduction and grinding of lignocellulosic materials. This process also provides mixing function if more than one type of waste will be involved for compost production. Following that, a Physical-Chemical Pretreatment is provided mainly for the loosening of fibrous materials and preconditioning of the materials for the following treatment. The preconditioning operations include Wetting and Debonding Agents addition, moisture adjustment, preheating, and mixing of materials. Major functions of the next process, High-Rate Stabilization Process, are decomposition of easily decomposable organics and removal of heavy metals, if any. Suitable oxidant(s) and metal extraction agents are added into high temperature and high pressure environments as discussed previously to achieve the objectives. In this High-Rate Stabilization Process other benefits such as further disassociation of fibrous materials, conversion of some cellulosic materials to humic substances, sterilization of pathogens and parasites, detoxification of organics, provision of operation energy can also obtained. The next step, High-Rate Activation Process, is provided to further improve characteristics (adsorption of nutrients, absorption of water, air and water circulating, thermal insulation, etc.) of the materials for compost production. Sterilization and detoxification effects are also existed in this process. Saturated steam at relatively high temperature and pressure, as well as Fluffing and Activation Agents are added into this process to assist achieving the anticipated objectives. Steam explosion operation is the final activity of the High-Rate Activation Process to further disassociation of fibrous materials. After the above operational processes a Product Refining Process is provided for moisture, particle size and nutrient conditioning to meet regulatory and market requirements. Product Bagging and Storage Facilities may be provided to complete the overall operation.

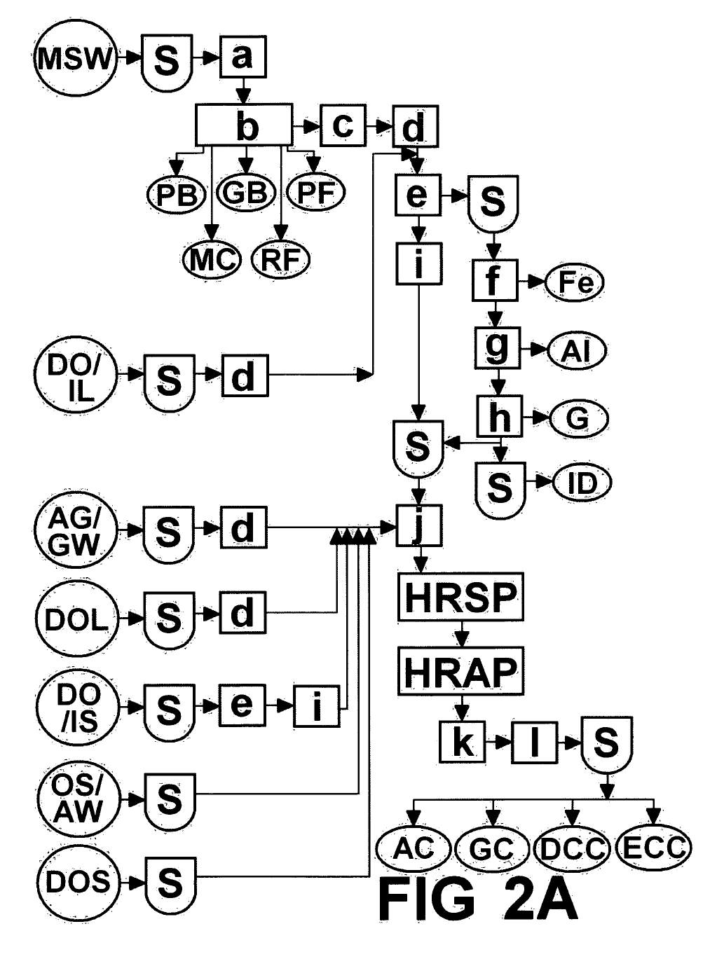

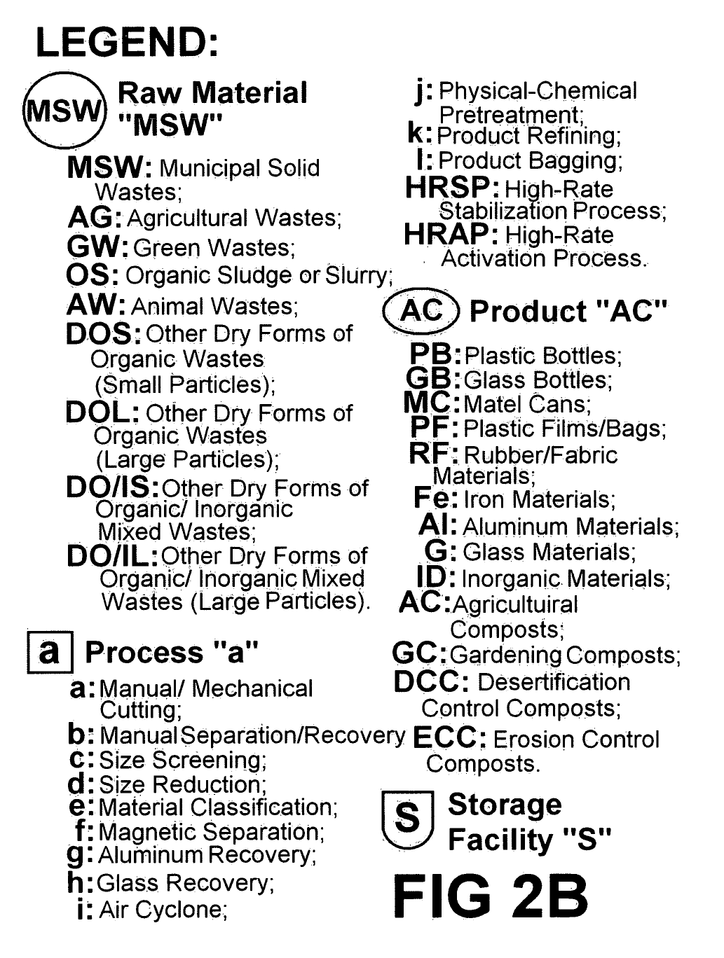

FIG. 2 shows overall flow charts of the compost production systems for different types of organic wastes. Legends are provided in the figure to explain types of waste, types of process, and types of products involved. As shown in FIG. 2, only MSW may require extensive preprocessing for inorganic and refractory organic materials separation and removal, by using Processes a, b, c, d, e, f, g, h and i as indicated on the Figure, where:
Process a=manual and/or mechanical cutting for plastic bags;
Process b=manual separation/recovery;
Process c=size screening (such as using trommels);
Process d=size reduction (such as using hammermills, grinders, and shredders);
Process e=organic/inorganic separation (such as using air classifiers, inertial separation,
   and air knife classifier);
Process f=ferrous metal recovery;
Process g=aluminum recovery;
Process h=glass recovery; and
Process i=recovery of organics (such as using air cyclone).
   Equipments used for the above processes are commercially available and are not included in this invention. However, for MSW, the above process flow sequences will be required for compost production, and will be incorporated into the subject invention. After the above operations, recovered organics from MSW will mainly include easily and moderately decomposable organics rich in cellulosic materials. These materials can be treated alone or mixed with other waste material(s) such as municipal treatment plant sludge, etc. as shown in FIG. 2. Again, other types of organic wastes listed in FIG. 2 can be treated alone or mixed together for compost production. In order to improve quality of compost products, mixing of wastes containing low cellulosic materials with wastes containing high cellulosic materials will be a good operation practice. On the contrary, mixing wastes containing low nutrient contents with wastes containing high nutrient contents will be a good operation practice also. The best choice would be mixing wastes containing high nutrient contents with wastes containing high celluloses contents. FIG. 2 is provided as examples for such operation practices. Different operation practices shall be developed depending on types of wastes to be treated based on principle as mentioned above.

Any organic wastes with large particle sizes require size reduction before entering into the Physical-Chemical Pretreatment Process (Process j indicated in FIG. 2). For example, if sizes are greater than sizes required for intended applications then Process d will be needed, such as composts for agricultural application then sizes smaller than about 12 mm would be desirable. If wastes containing both organic and inorganic materials a proper size reduction (Process d) and a separation (Process e) step are required, before entering into Process j (Physical-Chemical Pretreatment Process). The Process j operation includes Wetting Agent and/or Debonding Agent addition, mixing of different wastes, preheating, and moisture preconditioning.

After Process j, the High-Rate Stabilization Process (HRSP) (refer to FIG. 2) is provided for high rate stabilization of easily biodegradable organics and heavy metals. In HRSP, hydrolysis and oxidation reactions can cause further disassociation of lignocelluloses; high temperature can sterilize pathogens and parasites very effectively; high temperature, high pressure, existence of oxidants and high contact opportunities created by the HRSP reactor can destruct toxic organic compounds effectively; energy (steam) generated from oxidation of the easily decomposable organics can provide operation energy for other processes such as Physical-Chemical Pretreatment (Process j), and High-Rate Activation (HRAP) Processes to reduce operation costs.

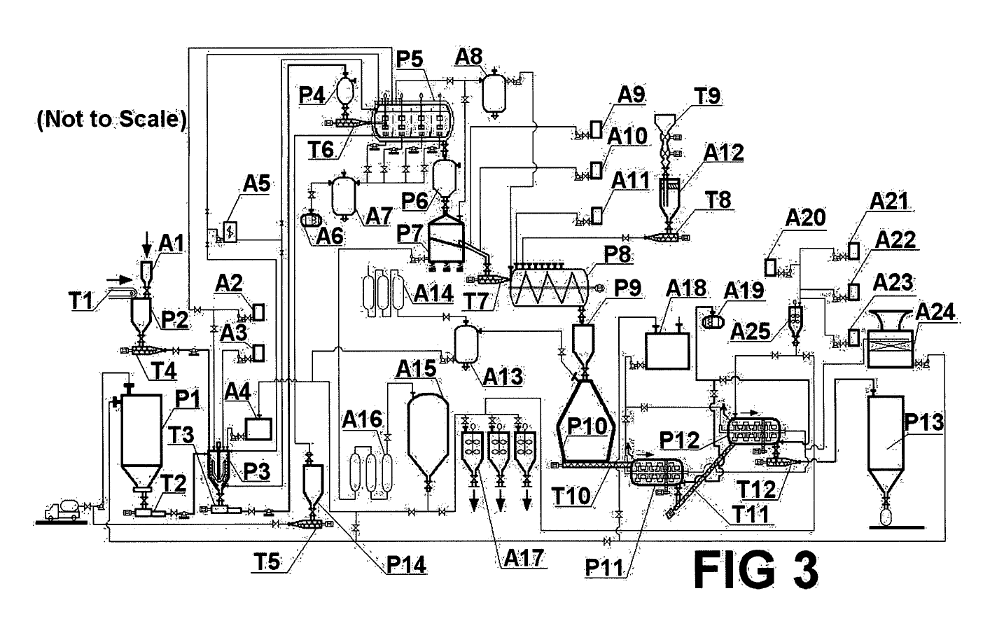

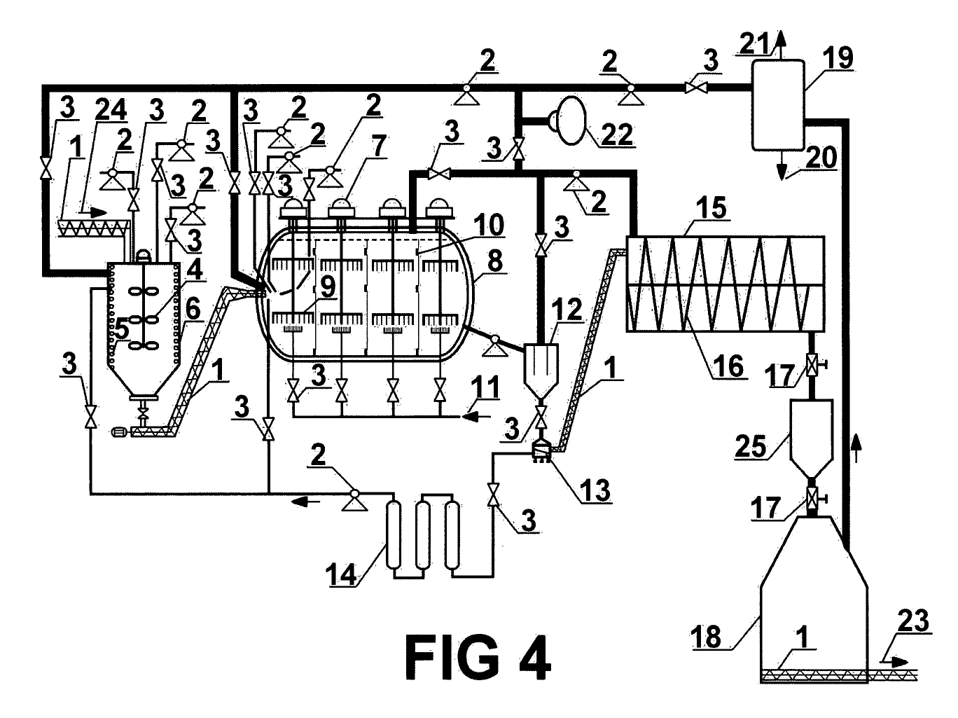

In between HRSP and HRAP a pressure reduction apparatus and a water/solid separation apparatus are needed (to be further discussed in FIGS. 3 and 4). Steam generated from pressure reduction can be used in the HRAP reactor for further treatment of celluloses and steam explosion purposes. Water separated from the solid materials can be treated for heavy metal removal, if necessary. This hot water can be partly recycled to Process j for preheating, and partly conditioned as a liquid fertilizer. As details in FIGS. 3 and 4 a vibration screen can be used to reduce water contents of the solid materials down to the Field Capacity. In HRAP reactor Fluffy and Activation Agents as discussed previously can be added to the compost materials to further improve the characteristics of the compost. After that the Product Refining Process is provided for conditioning of moisture, particle sizes, and nutrient contents.

FIG. 3 shows a typical P&ID (Processes and Instrumental Diagrams) of a composting plant using the subject invention. FIG. 3 illustrates a typical sludge composting plant for municipal treatment plant sludge or other types of wastewater treatment plant sludge. In order to increase the cellulosic contents of the compost produced, sludge can be mixed with selected agricultural, green, or processed MSW wastes. Explanation of the apparatus used are provided below. Apparatus are grouped into three types: P (process equipment), T (transfer equipment) and A (auxiliary equipment).

### Process Equipment:

P1: Sludge or slurry storage tank;
P2: Dry Input Waste(s) Storage Tank;
P3: Physical-Chemical Pretreatment Tank;
P4: No. 1 Equalization Tank;
P5: High-Rate Stabilization Reactor;
P6: No. 2 Equalization Tank;
P7: Vibration Separator;
P8: High-Rate Activation Reactor;
P9: No. 3 Equalization Tank;
P10: Steam Explosion Tank;
P11: No. 1 Product Refining Reactor;
P12: No. 2 Product Refining Reactor;
P13: Product Bagging Equipment; and
P14: Material Return Flow Tank.

### Transfer Equipment:

T1: Shredded Waste Conveyor;
T2: No. 1 Transfer Pump;
T3: No. 2 Transfer Pump;
T4: No. 1 Screw Conveyor;
T5: No. 2 Screw Conveyor;
T6: No. 3 Screw Conveyor;
T7: No. 4 Screw Conveyor;
T8: No. 5 Screw Conveyor;
T9: Rotary Air-Lock Conveyor;
T10: No. 6 Screw Conveyor;
T11: No. 7 Screw Conveyor; and
T12: No. 8 Screw Conveyor.

### Auxiliary Equipment:

A1: Wetting Agent Storage Tank;
A2: Metal Extraction Agent Storage Tank;
A3: Debonding Agent Storage Tank;
A4: Dilution Water Storage Tank;
A5: Electric Oil Furnace;
A6: No. 1 Air Compressor;
A7: Compressed Air Storage Tank;
A8: Steam Storage Tank;
A9: Neutralization Agent Storage Tank;
A10: Reducing Agent Storage Tank;
A11: Fluffing Agent Storage Tank;
A12: Activation Agent Storage Tank;
A13: Condensation Tank;
A14: Air Purification Columns;
A15: Hot Water Storage Tank;
A16: Water Treatment Columns;
A17: Liquid Fertilizer Storage Tanks;
A18: Cooling Water Storage Tank;
A19: No. 2 Air Compressor;
A20: Micronutrient Storage Tank;
A21: N-Compound Storage Tank;
A22: P-Compound Storage Tank;
A23: K-Compound Storage Tank;
A24: Cooling Tower; and
A25: Nutrient Mixing Tank.
Besides the above major equipments, numerous other apparatus are also involved in the subject sludge treatment plant, such as valves, and instruments (temperature, pressure, pH, Eh, DO, etc.). Major functions of each major apparatus are either self-explanatory or explained previously already.

FIG. 4 presents an example of details of the most important three major apparatus (i.e., Physical-Chemical Pretreatment Tank, High-Rate Stabilization Reactor, and High-Rate-Activation Reactor) and their associated apparatus used in this invention. Explanation of numerical numbers on FIG. 4 is provided as follows:
1: Screw conveyor;
2: Pump;
3: Control valve;
4: Mixer;
5: Heat exchanger;
6: Physical-Chemical Pretreatment Tank;
7: Motor;
8: High-Rate Stabilization Reactor,
9: Disc Type Mixer;
10: Control gate;
11: Air or oxidant(s);
12: Cyclone separator;
13: Vibration screen;
14: Metal removal apparatus (e.g., ion-exchangers, precipitator, etc.);
15: High-Rate Activation Reactor;
16: Screw Steamer;
17: Steam explosion control valve;
18: Steam explosion Collector;
19: Steam-water separator;
20: Condensate;
21: Steam;
22: Steam Generator;
23: Solid materials (to be transferred to the product refining process); and
24: Input wastes (from mechanical pretreatment process).
   Again, all apparatus listed above are explained previously in this invention. The foregoing description is intended to illustrate various aspects of the present invention. It is not intended that the examples presented herein limit the scope of the present invention. The invention system and major apparatus are fully described above, it will be apparent to one of ordinary skill in the art that many changes and modifications can be made thereto without departing from the spirit or scope of the appended claims.

I claim:

1. A high-rate thermal-chemical-mechanical method and apparatus for the direct production of high quality activated composts or organic fertilizers from organic types of wastes or materials, comprising the processes of:
(a) Waste Screening Processes: These processes use various types of solid waste separation apparatus to separate and recycle inorganic materials and refractory organics from the waste streams. Easily and moderately decomposable organics are screened out for the following processes.
(b) Mechanical Pretreatment Processes: These processes use size reduction apparatus to reduce the sizes of organic wastes to more uniform sizes for further purification. These processes also provide operations for pretreatment (disassociation) of lignocellulosic materials and for mixing of different types of organic wastes, if any.
(c) Physical-Chemical Pretreatment Process: Organic waste materials from the above processes are mixed with appropriate Wetting Agent(s), Debonding Agent(s), and are adjusted to suitable moisture contents and pre-heated for the following process.
(d) High-Rate Stabilization Process: This process is conducted under suitable temperature and pressure conditions, maintaining materials in solid and liquid phases in the reactor, avoiding the formation of gas phases as much as possible, adding suitable oxidants and heavy metal extracting agents, when necessary, to oxidize easily decomposable organics by partial wet oxidation process and to extract heavy metals, when necessary, from the inputting wastes. Through thermal hydrolysis and oxidation reactions, portions of the disassociated cellulosic (fibrous) materials are also transformed to humic substances. After reactions the materials are subjected to dewatering and partial decompression.
(e) High-Rate Activation Process: Reducing, Neutralization, Fluffing and Activation Agents are added to the reactor when necessary under suitable high temperatures and pressures maintained by saturated steam in the reactor. After activation reactions a steam explosion process is conducted to further improve the activation characteristics of the materials.
(f) Product Refining Processes: Based on regulatory requirements and market needs, the above processed materials are subject to moisture, size and nutrient adjustments.

2. The organic types of wastes or materials as defined in claim 1 suitable for the direct production of activated composts or organic fertilizers by the subject method and apparatus include municipal solid wastes (MSW), agricultural wastes, green wastes, wastewater treatment plant sludge, animal wastes, some types of organic wastes from manufacturing plants such as food processing, paper manufacturing, refinery, and medicine manufacturing plants, organic wastes from institutional facilities, as well as wastes from landfill mining (cleanup), and any types of waste containing significant amount of easily decomposable materials such as proteins, lipids and certain carbohydrates, etc. as well as moderately decomposable materials such as lignocellulosic materials, and its disassociated materials such as celluloses, hemicelluloses, and lignin materials.

3. The method and apparatus as defined in claim 1 comprising the following major types of equipments and operational parameters:
(a) Waste Screening Processes: Waste Screening Processes are operated indoor with negative pressure to avoid odor dissipation problems. Air extracted from the indoor building is treated by processes such as activated carbon, scrubbing, chemical oxidation and other air purification methods. Waste Screening Processes can be practiced partly manually and partly by machine, or totally by machine depending on incoming waste characteristics, and cost-effectiveness. In the Waste Screening Processes the incoming wastes can be processed and purified by machines such as vibrating screens, trommels, disc screens, etc. for inorganic removal, when necessary.
(b) Mechanical Pretreatment Processes: Incoming wastes are shredded and grinded to relatively uniform sizes for organics/inorganics separation and recycling. Sizes can be selected between 0.05 mm to 5 cm depending on intended uses of the composting products by equipments such as hammermills, grinders, and shredders. After size reduction to more uniform sizes, organics and inorganics in the incoming wastes can be further separated and purified by equipments such as air classifiers, inertial separation, and air knife classifier. Suitable Wetting and/or Debonding agents can also be added right before size reduction operation to enhance disassociation of lignocelluloses and energy saving for size reduction operation.
(c) Physical-Chemical Pretreatment Process: Pretreated organic materials from the Mechanical Pretreatment Processes and other types of organic wastes which do not require size reduction and separation operations are transferred into this process to adjust the moisture contents to 0% to 20% above saturation conditions, and to add 0.5% to 5% of Wetting Agent(s), 0.5% to 5% Debonding Agent(s), when needed. Waste heat from the High-Rate Stabilization and Activation reactors can be used to pre-heat the materials in this process to save energy.
(d) High-Rate Stabilization Process: Under conditions of temperature between 140° C. to 300° C. and pressure between 7 to 88 atm, avoiding the formation of gas phases as much as possible by maintaining at least 50 to 100 psi (approximately 3.5 to 7 atm) of pressure higher than the corresponding saturated water vapor pressure generated by the temperature in the reactor, adding 1 to 6 ppm equivalent of dissolved oxygen contents of oxidant(s) in the aqueous phase, and 0.5% to 10% of heavy metal extraction agent(s) in the aqueous phase, when needed, to react for 5 to 30 minutes. Both horizontal or vertical reactors can be used for the above reactions. The High-Rate Stabilization Reactor is designed to reduce the short-circuiting effects with multiple compartments or other means. Reactor is designed in a way that spontaneous and continuous partial oxidations will occur beyond the temperatures of self-sustaining reaction temperatures. After reactions, treated materials are subject to a partial decompression to reduce pressure down to the saturated steam pressure by an equalization tank and dewatering to near the field capacity by a vibrating separator or equivalent. Steam generated from the decompression is diverted to the High-Rate Activation Reactor. Aqueous solution generated is partly transferred to the Physical-Chemical Pretreatment Tank for pre-heating and dilution, and partly used for the production of liquid fertilizers. The dewatered materials are transferred to the High-Rate Activation Reactor.
(e) High-Rate Activation Process: Sufficient amount of Reducing and Neutralization Agents are added to the inputting materials to remove extra oxidant(s) in the materials and adjust pH to near neutral conditions. 0.5 to 5% and 1 to 50% of Fluffing and Activation Agents, respectively, are added to the inputting materials. Types of reactors can be used for this process include auger digesters, rotary kilns, and autoclaves. Temperatures of the reactor are in 140° C. to 300° C. or obtained from the previous processes depending on types of wastes treated. Pressures of the reactor are maintained by the saturated steam at the corresponding temperatures. After 5 to 30 minutes of reaction time period, materials in the reactor are subject to a decompression operation and the pressure reduced to the atmospheric pressure. The decompressed materials are then transfer to the Product Refining Processes.
(f) Product Refining Processes: Centrifuge and/or heat exchange reactors are used to adjust the moisture contents to the levels (usually less than 35% moisture contents by weight) required by regulatory agencies and market needs. Size reduction equipments as mentioned above in claim 3(b) are used to adjust the particle sizes to requirements by regulatory agencies and market needs. Based on market needs or for the purpose of increasing product values, NPK chemical compounds are added, when needed.

4. As defined in claims 1 and 3, for the direct production of high quality activated composts or organic fertilizers by high-rate composting methods, any of the following two types of Wetting Agents are used: organic and inorganic types. Inorganic Wetting Agents presented by this invention include expansible clay minerals (such as montmorillonite, especially sodium montmorillonite, or called bentonite, and kaolinite, vermiculite, perlite, etc.) and multi-valenced and positively ionized metallic compounds which can infiltrate into negatively charged fibers (such as alum or aluminum sulfate, titanium dioxide, etc.). The inorganic Wetting Agents can be used for the subject invention also include chemical compounds which can assist expansion and softening of fibers, such as carbonates (sodium carbonate, magnesium carbonate, calcium carbonate, ammonium carbonate, etc.), and bicarbonates (sodium bicarbonate, ammonium bicarbonate, etc.). Organic Wetting Agents can be used for the subject composting process include various types of fatty acid esters, and non-ionic surfactants. Examples of fatty acid esters are glycerol monostearate, glycerol monooleate, diethylene glycol monostearate, diethylene glycol monooleate, propylene glycol monooleate, etc. Among them fatty acids of alcohols containing at least one ether group are more suitable to use for the subject purpose, such as diethylene glycol, triethylene glycol, polyethylene glycol, and polypropylene glycol. Non-ionic surfactants can be used for the subject composting as Wetting Agents are commercially available such as Triton X-100, Triton X-45, Triton X-114, etc.

5. As defined in claims 1 and 3, for the direct production of high quality activated composts or organic fertilizers by high-rate composting methods, cationic quaternary ammonium compounds can be used as Debonding Agents. Examples of these types of compound include trimethylalkyl ammonium halides, trimethylalkylene ammonium halides, methylpolyoxyethylene alkylene ammonium halides, etc. as shown in the following common formula: 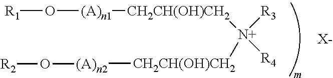
Where: R₁ and R₂=aliphatic hydrocarbons with 12 to 40 carbons;
R₃ and R₄=methyl, ethyl, hydroxyethyl groups;
A=oxyalkylene group, derived from both ethylene oxide and propylene oxide;
m=a number corresponding to the valence of X;
ₙ₁ and ₙ₂=average number of oxyalkylene units (6 to 30);
X=anion.
Other Debonding Agents such as mixtures of a phospholipids, a non-ionic surfactant, and optionally a lubricating additive are used in this invention.

6. As defined in claims 1 and 3, for the direct production of high quality activated composts or organic fertilizers by high-rate composting methods, one or combinations of the following oxidants can be selected by this invention for composting: ozone, chlorine, hypochlorites, potassium permanganate, hydrogen peroxide, oxygen, and air.

7. As defined in claims 1 and 3, for the direct production of high quality activated composts or organic fertilizers by high-rate composting methods, one or combinations of the following Heavy Metal Extraction Agents are used: NH4Ac, Ca(NO3)2, Mg(NO3)2, MgCl2, NH4Ac+NH4OH (pH=9), 2% citric acid, 0.1N HCl, 0.2M ammonium oxalate, EDTA, 1N NH4Ac+0.2% hydroquinone, NH2OH.HCl, 0.04M to1M NH2OH.HCl+25% HAc, sodium dithionite-sodium citrate, dilute acids, NH2OH.HCl+dilute acids, H2O2, sodium hypochlorite, H2O2+dilute acids, ozone, chlorine, other hypochlorite salts, potassium permanganate, oxygen, above listed oxidants plus dilute acids, strong acids, and mixtures of strong acids, especially HNO3+HF+HClO4.

8. As defined in claims 1 and 3, for the direct production of high quality activated composts or organic fertilizers by high-rate composting methods, this invention presents three types of Fluffing Agents for achieving the above stated compost quality improvements:
(a) Add cationic retention agents and anionic or non-ionic surfactants to dry incoming wastes right before size reduction operation (i.e., right before the Mechanical Pretreatment), or after cellulosic pulp formation stage (i.e., the final stage of the High-Rate Stabilization Process).
(b) Add mixtures of phospholipids, a non-ionic surfactant, and optionally a lubricating additive right before size reduction operation or after High-Rate Stabilization Process.
(c) Add an antioxidant and hydrophilic agent, as well as an inorganic swelling chemical right before the final stage of the steam explosion operation (i.e., after the High-Rate Activation Process).
Chemicals for the type (a) agents listed above include aluminum sulfate (as a cationic retention agent) plus paraffin (as a non-ionic surfactant), or cationic quaternary ammonium compounds plus nonionic fatty acid esters. Phospholipids in the type (b) agents include phosphatidylcholine or lecithin, hydroxylated phosphatidylcholine, phosphatidylethanolamine, etc. Non-ionic surfactants mentioned in the type (b) above are similar to that used for Wetting Agents such as diethylene glycol, triethylene glycol, polyethylene glycol, and polypropylene glycol and Triton X-100, Triton X-45, Triton X-114, Igepal CO-630, Igepal CO-430, etc. Plant oils such as olive oil, caster oil and other vegetation oils are candidates for lubricating additives. Type (c) mentioned above are mostly inorganic compounds such as Na₂SO₃, K₂SO₃, MgSO₃, and (NH₄)₂SO₃ for antioxidant and hydrophilic agents, and MgCl₂, Na₂CO₃, NaHCO₃, (NH₄)₂CO₃, MgCO₃, and NH₄HCO₃ for swelling chemicals. Among the above chemical candidates, this invention suggests that type (c), especially those chemicals also have nutrient ingredients shall be priority candidates due to the reason that, besides the advantages of adding Fluffy Agents, the compost nutrient contents can be increased and many of the least soluble metal solid species (such as carbonates of Cd, Cu, Ni, Pb, and Zn) can be also formed to reduce the impacts by heavy metals, if any.

9. As defined in claims 1 and 3, for the direct production of high quality activated composts or organic fertilizers by high-rate composting methods, one or combinations of the following Activation Agents can be used: clay minerals (bentonite, kaolinite, vermiculite, perlite, zeolite, etc.), activated carbon, peat, and brown coal.

10. As defined in claims 1 and 3, for the direct production of high quality activated composts or organic fertilizers by high-rate composting methods, the operation system includes the following major process and instrumental equipments and major operational steps: The purified and size reduced dry organic wastes are transferred to a Dry Wastes Storage Tank (P2) through a conveyor (T1). Wetting Agent(s) can be added from a storage tank (A1) to the Dry Wastes Storage Tank (P2) for pre-mixing. Sludge, slurry or any wet types of small particle sizes organic wastes are received by a Sludge Storage Tank (P1). Wastes from the Dry Wastes Storage Tank (P2) are transferred to the Physical-Chemical Pretreatment Tank (P3) by a screw conveyor (T4). Wet wastes from the Sludge Storage Tank (P1) are transferred to the Physical-Chemical Pretreatment Tank (P3) by a Transfer Pump (T2). Mixing ratio of wastes received by the Physical-Chemical Pretreatment Tank (P3) are based on anticipated final cellulosic contents, nutrient contents, and overall heat contents in the easily decomposable organic fraction in the mixed wastes. The Physical-Chemical Pretreatment Tank (P3) is equipped with mixers and heat-exchange coils. Debonding Agent(s) can be added from the Debonding Agent Storage Tank (A3) to the Physical-Chemical Pretreatment Tank (P3). The Physical-Chemical Pretreatment Tank (P3) also provides input accesses for Heavy Metal Extraction Agent(s) from Metal Extraction Agent Storage Tank (A2), and for dilution water from the Dilution Water Storage Tank (A4). The processed materials in the Physical-Chemical Pretreatment Tank are transferred to a No. 1 Equalization Tank (P4) by a transfer pump (T3). The materials in the No. 1 Equalization Tank (P4) are then transferred to the High-Rate Stabilization Reactor (P5) by a Screw Conveyor (T6) based on a calculated rate which can maintain the selected detention time of the High-Rate Stabilization Reactor (P5). The High-Rate Stabilization Reactor (P5) is divided into 1 to 8 compartments with a disc type of mixer in each compartment. Oxidizer(s) are injected into the High-Rate Stabilization Reactor (P5) from an Oxidizer Storage Tank (A7). If air is used as an oxidant, an Air Compressor (A6) is provided. After processing by the High-Rate Stabilization Reactor (P5), processed materials are transferred to a No. 2 Equalization Tank (P6) from the last compartment of the High-Rate Stabilization Reactor (P5). Through partial decompression, materials from the No. 2 Equalization Tank (P6) are pressurized to the Vibration Separator (P7) for dewatering. The separated hot water is treated for heavy metal removal, if needed, by Water Treatment Columns (A16), and stored in a Hot Water Storage Tank (A15). A cooling water device may be used before Liquid Fertilizer Storage Tank (A17). Superheated steam generated from the Vibration Separator (P7) and the High-Rate Stabilization Reactor (P5) is stored in a Steam Storage Tank (A8). This superheated steam is pressurized to the High-Rate Activation Reactor (P8). Dewatered solid materials from the Vibration Separator (P7) are transferred to the High-Rate Activation Reactor (P8) by a Screw Conveyor (T7). Neutralization and Reducing Agents are added to the dewatered materials from the Neutralization Agent Storage Tank (A9) and the Reducing Agent Storage Tank (A10) during transfer operation. Fluffing and Activation Agents are injected into the High-Rate Activation Tank (P8) from the respective storage tanks (A11 and A12). After treatment in the High-Rate Activation Reactor (P8), steam explosion operation is processed by a No. 3 Equalization Tank (P9) and a Steam Explosion Tank (P10). If the Activation Agent(s) are in dry powdered or granular forms, a doubled Rotary Air Lock Conveyor (T9) is used for pressurized transfer of the Activation Agent(s). Steam exploded materials in the Steam Explosion Tank (P10) are then transferred by a Screw Conveyor (T10) to a series of Product Refining Reactors (P11 and P12) for cooling, size refining, moisture and nutrient adjustments by cold water supplying from a Cooling Water Storage Tank (A18), cold air from an Air Compressor (A19), micronutrients from a Micronutrient Storage Tank (A20), and N, P, and K compounds from their respective storage tanks (A21, A22 and A23). A Nutrient Mixing Tank (A25) is provided for preparation of selected amounts of N, P and K compounds. Commercially available product bagging equipment(s) (P13) are provided for compost bagging operation.

11. Composts or organic fertilizers produced by methods and equipments defined in claims 1 and 3 are termed “Activated Composts” by this invention due to the enhancement/activation of six major compost characteristics way beyond that of the composts produced by the traditional biochemical processes. These six major compost characteristics which can enhance the compost quality are: (a) moisture absorption and holding capability, (b) nutrients adsorption and holding capability, (c) soil particles holding and conserving capability, (d) soil air ventilation capability, (e) soil water transmission capability, and (f) soil thermal insulation capability.
